(19) United States
(12) Patent Application Publication
(10) Pub. No.: US 2025/0332298 A1
(43) Pub. Date: Oct. 30, 2025

(54) PEPTIDE-COUPLED ALGINATE GELS COMPRISING RADIONUCLIDES

(71) Applicant: BLUE WAVE THERAPEUTICS GMBH, Bäch SZ (CH)

(72) Inventors: Michael DORNISH, Bekkestua (NO); Jostein DAHLE, Bekkestua (NO)

(21) Appl. No.: 18/703,281

(22) PCT Filed: Oct. 19, 2022
(86) PCT No.: PCT/EP2022/079090

§ 371 Date: Apr. 19, 2024

(30) Foreign Application Priority Data
Oct. 21, 2021 (NO) 20211268

(51) Int. Cl. A61K 51/08; A61K 51/06; A61K 51/12

(52) U.S. Cl. CPC A61K 51/082; A61K 51/065; A61K 51/1213; A61K 51/1255

(57) ABSTRACT

The present invention relates to alginate-based gels for use in radiotherapy. More particularly, the invention provides an alginate gel comprising a peptide-coupled alginate, at least one type of divalent cation, and at least one radionuclide cation; a method for providing at least one alginate gel particle; a composition comprising said alginate gel; said alginate gel or composition for use as a medicament; said alginate gel or composition for use in a method for the treatment of a proliferative disease; and kits comprising an alginate and a radionuclide cation.

### FIELD OF THE INVENTION

The present invention relates to alginate gels for use in radiotherapy. More particularly, the invention provides an alginate gel comprising a peptide-coupled alginate, at least one type of divalent cation, and at least one radionuclide cation; a method for providing at least one alginate gel particle; a composition comprising said alginate gel; said alginate gel or composition for use as a medicament; said alginate gel or composition for use in a method for the treatment of a proliferative disease; and kits comprising an alginate and a radionuclide cation.

### REFERENCE TO AN ELECTRONIC SEQUENCE LISTING

The sequence listing (142001_221221_Peptide-Coupled_Alginate_Gels_Comprising_Radionuclides-3-corr.xml; Size: 30,265 bytes; and Date of Creation: Sep. 6, 2024) is herein incorporated by reference in its entirety.

### BACKGROUND OF THE INVENTION

Alpha and beta particle emitting radionuclides have unique properties that make them attractive for use in therapy against various diseases such as cancer. However, the delivery of the radionuclides may be challenging.

It has been previously suggested that alpha emitters can be used bound to particulates and colloids for internal radionuclide therapy. However, many colloidal and ceramic particulate and microsphere formulations represent either non-biocompatible or non-biodegradable formulations, as the microspheres are toxic or cause an immunogenic reaction. The toxicity and immunogenic reactions last typically for an extended period of time after the radiation has decayed, since the particles cannot be degraded easily.

Successful delivery of the radiation to unwanted cells depends on a stable radioisotope-carrier interaction to prevent unanticipated side effects due to radioisotope leakage as well as specific targeting of the radioisotope-carrier to the unwanted cells. A problem with prior art formulations is that the radionuclide may be released from the composition, and/or that daughter nuclides formed from radioactive decay of the initially incorporated radionuclide may not bind to the carrier. The consequence is unwanted off-target toxicity when the released radionuclides diffuse or are transported away from the target. Another problem with prior art formulations is that many rely on unspecific binding to structures, such as bone, close to the unwanted cells or local application close to the unwanted cells to achieve targeting.

Hence, there is a need for improved drugs for radiotherapy.

### BRIEF DESCRIPTION OF THE DRAWINGS

FIG. 1 illustrates chelation of cations by alginate.

FIG. 2 shows a photomicrograph of alginate gel microspheres.

FIG. 3 shows the results from measurements of the radioactivity from radium-223 bound to calcium crosslinked, homogeneous gelled alginate microspheres over time.

FIG. 4 shows the results from measurements of the radioactivity from lead-211 bound to calcium crosslinked, homogeneous gelled alginate microspheres over time.

FIG. 5 shows the results from measurements of the radioactivity from bismuth-211 bound to calcium crosslinked, homogeneous gelled alginate microspheres over time.

FIG. 6 shows the results from measurements of the radioactivity from radium-223 bound to strontium crosslinked, inhomogeneous gelled alginate microspheres over time.

FIG. 7 shows the results from measurements of the radioactivity from lead-211 bound to strontium crosslinked, inhomogeneous gelled alginate microspheres over time.

FIG. 8 shows the results from measurements of the radioactivity from bismuth-211 bound to strontium crosslinked, inhomogeneous gelled alginate microspheres over time.

FIG. 9 shows the results from measurement of the radioactivity from actinium-225 as well as from decay daughter radionuclides francium-22 and bismuth-213 bound to strontium crosslinked, homogeneous gelled alginate microspheres over time.

FIG. 10 shows the results from measurements of the radioactivity from radium-223 bound to barium crosslinked, inhomogeneous gelled alginate microspheres over time.

FIG. 11 shows the results from measurements of the radioactivity from lead-211 bound to barium crosslinked, inhomogeneous gelled alginate microspheres over time.

FIG. 12 shows the results from measurements of the radioactivity from bismuth-211 bound to barium crosslinked, inhomogeneous gelled alginate microspheres over time.

FIG. 13 shows the results from measurements of the radioactivity from actinium-225 and its decay daughter radionuclides francium-221 and bismuth 213 in solution and that bound to calcium alginate microparticles over time.

FIG. 14 shows the results from measurements of the radioactivity from addition of different amounts of actinium-225 bound to calcium alginate microparticles over time.

FIG. 15 shows the results from measurements of the radioactivity from addition of different amounts of francium-221 bound to calcium alginate microparticles over time.

FIG. 16 shows the results from measurements of the radioactivity from addition of different amounts of bismuth-213 bound to calcium alginate microparticles over time.

FIG. 17 shows the weight of different alginate gels over time.

FIG. 18 shows the Mean Channel Number versus the concentration of FITC-RGD peptide added to MDCK cells in an experiment related to binding of said peptide to cells.

FIG. 19 shows the changes in cell number over time for cells added different alginate gels.

### BRIEF SUMMARY OF THE INVENTION

In one aspect, the present invention relates to an alginate gel as claimed in claim 1,
wherein the alginate gel comprises
   an alginate comprising at least one peptide having a peptide sequence for interacting with a receptor of a cell affected by a proliferative disease;
   at least one type of divalent cations selected from the group comprising Ba²⁺, Sr²⁺, Ca²⁺, Cu²⁺, Zn²⁺, and combinations thereof; and
   at least one radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof,
   wherein the alginate chelates the divalent cations and the at least one radionuclide cation.

Advantageously, the gel is able to bind radionuclide cations by chelation, while the peptide sequence provides selectivity for certain cells.

In another aspect, the invention relates to a method for providing at least one alginate gel particle comprising an alginate comprising at least one peptide having a peptide sequence for interacting with a receptor of a cell affected by a proliferative disease and at least one radionuclide cation, as claimed in claim 6, wherein the method comprises the steps of
i) providing a solution of an alginate, wherein the alginate comprises at least one peptide having a peptide sequence for interacting with a receptor of a cell affected by a proliferative disease;
ii) dripping the solution from step i) into an aqueous solution comprising a divalent cation, wherein said divalent cation is selected from Ca²⁺, Sr²⁺, and Ba²⁺, to form at least one alginate gel particle; and
iii) contacting the alginate gel particle of step ii) with a solution comprising at least one radionuclide cation.

In another aspect, the invention relates to a composition comprising an alginate gel as described above, as claimed in claim 7, together with at least one pharmaceutically acceptable carrier, diluent, and/or excipient.

In another aspect, the invention relates to a kit comprising,
in a first container,
an alginate gel comprising
   an alginate comprising
      at least one peptide having a peptide sequence for interacting with a receptor of a cell affected by a proliferative disease; and
      at least one type of divalent cations selected from the group comprising Ba²⁺, Sr²⁺, Ca²⁺, Cu²⁺, Zn²⁺, and combinations thereof; and
optionally, a suitable solvent; and
in a second container,
   at least one radionuclide cation, and
   a suitable solvent.

In another aspect, the invention relates to a kit comprising,
in a first container,
   a water-soluble alginate,
      wherein the alginate comprises at least one peptide having a peptide sequence for interacting with a receptor of a cell affected by a proliferative disease,
   water, and
   optionally, another suitable solvent;
in a second container,
   particles that are insoluble in water, said particles comprising an alginate and at least one type of divalent cation selected from the group comprising Ba²⁺, Sr²⁺, Ca²⁺, Cu²⁺, Zn²⁺, and combinations thereof, and
   water; and
in a third container
   at least one radionuclide cation, and
   a suitable solvent.

In another aspect, the invention relates to the gel or the composition described above, for use as a medicament, as claimed in claim 10.

In another aspect, the invention relates to the gel or the composition described above, for use in the treatment of a proliferative disease, as claimed in claim 11.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The inventors have discovered that alginate gels comprising an alginate comprising at least one peptide having a peptide sequence for interacting with a receptor of a cell affected by a proliferative disease may be used to stably chelate a radionuclide and selectively deliver radiation to unwanted cells. Radionuclide cations can compete with the gelling ions of the alginate gel for binding to the alginate and replace some of the original gelling ions, thereby themselves becoming part of the gel network. When a radionuclide cation is chelated by an alginate to form part of an alginate-based gel, the alginate gel may hold the radionuclide effectively and firmly in place by chelation. The peptide may provide important selectivity. Hence, such alginate gels comprising radionuclides represent promising drug candidates.

Thus, in one aspect, the invention relates to an alginate gel comprising a peptide-coupled alginate, wherein the peptide is a peptide for interacting with a receptor of a cell affected by a proliferative disease, and wherein the alginate gel further comprises a radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof.

In some embodiments, the invention relates to an alginate gel comprising
an alginate comprising at least one peptide having a peptide sequence for interacting with a receptor of a cell affected by a proliferative disease;
at least one type of divalent cations selected from the group comprising Ba²⁺, Sr²⁺, Ca²⁺, Cu²⁺, Zn²⁺, and combinations thereof; and
at least one radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof,
   wherein the alginate chelates the divalent cations and the at least one radionuclide cation.

Alginate, structure I, is a structural polysaccharide found in brown algae, comprising up to 40% of the dry matter. Its main function is to give strength and flexibility to the algal tissue. Alginate is an unbranched binary copolymer of (1→4)-linked β-D-mannuronic acid (M) and α-L-guluronic acid (G) residues. In the below representation of structure I, “G” and “M” identifies α-L-guluronic acid β-D-mannuronic acid, respectively. The numbering of the carbons is also indicated, as well as the type of glycosidic bond (α and β).

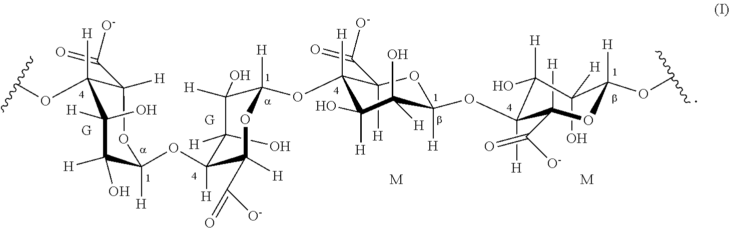

The relative amount of the two uronic acid monomers and their sequential arrangement along the polymer chain vary widely, depending on the origin of the alginate. The uronic acid residues are distributed along the polymer chain in a pattern of blocks, where homopolymeric blocks of guluronate (G) residues (G-blocks), homopolymeric blocks of mannuronate (M) residues (M-blocks) and blocks with alternating sequence of M and G units (MG-blocks) co-exist. Thus, the alginate molecule cannot be described by the monomer composition alone. NMR characterisation of the sequence of M and G residues in the alginate chain is needed in order to calculate average block lengths. It has also been shown by NMR spectroscopy that alginate has no regular repeating unit. The functional properties of alginate are primarily influenced by the G content, the average number of G's in a G-block length and the molecular weight.

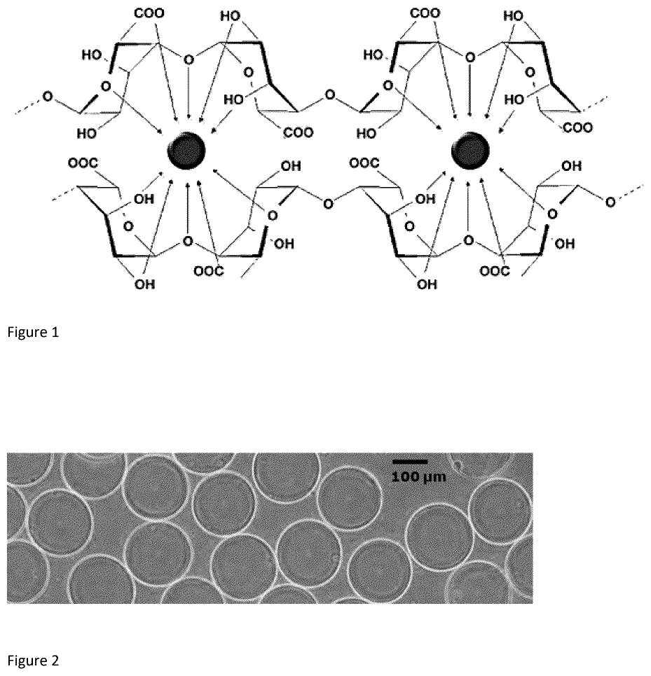

Alginates have a strong affinity for cations which decreases in the following order: Pb²⁺>Cu²⁺=Ba²⁺>Sr²⁺>Cd²⁺>Ca²⁺>Zn²⁺>Co²⁺>Ni²⁺. Alginate forms gels with most di-and multivalent cations, although calcium is most widely used. Cations used to form an alginate gel are referred to as “gelling ions”. Most monovalent cations and the divalent Mg²⁺ ions do not induce gelation, while ions like Ba²⁺ and Sr²⁺ will produce stronger alginate gels than Ca²⁺. The gelling reaction, i.e. the formation of the alginate-based gel, occurs when cations take part in interchain binding between G-blocks within the alginate molecules giving rise to a three-dimensional network in the form of a gel. As illustrated in FIG. 1, cations, which are shown in FIG. 1 as dark spheres, and which may be referred to as gelling cations, are effectively chelated by alginate through ionic interactions between the cation and lone-pair electrons of oxygen atoms in the hydroxyl groups and in the glycosidic bond. Consecutive guluronic residues, for instance, have the ability to cross-link with cations. Particularly calcium, strontium, and barium effectively cross-link alginate polymer chains forming gels.

Gelation with, for instance calcium ions, results in the instantaneous formation of heat-stable gels that can be formed and set at room temperature and at physiological pH's. The gel strength will depend upon the guluronic content and on the average number of G-units in the G-blocks. In addition, using alginates with increasing molecular weights will also increase the strength of the gel, at least up to a certain limit of molecular weight. A high G content generally results in a stronger, stiffer, more brittle and more porous gel. Conversely, high M content results in gels which are more elastic and weaker.

The invention provides an alginate gel. The term “gel” as used herein is intended to mean a three-dimensional network organisation which has the ability to be interpenetrated by a liquid, in which the structural coherent matrix may contain a high portion of liquid. The gel may comprise said liquid, or it may be dry. A “dry” gel may have been prepared by drying a “wet” gel, or it may have been obtained in any other manner known to the skilled person, such as by precipitation. The gel may be in different forms, including but not limited to block gels, foams, pastes, amorphous gels, and particles. When water is the solvent, the gel may be defined as “hydrogel”.

It is known from the literature that the biopolymer alginate may occur as mannuronate-rich or guluronate-rich polymer, i.e. the percentage composition of alginate is greater than 50% mannuronic acid in mannuronate-rich alginate while the percentage composition is greater than 50% guluronic acid in guluronate-rich alginate. G-rich alginate has a greater percentage of guluronic acid residues and may have a higher number of consecutive guluronate moieties in a series, i.e. the G-block size. It is further known that G-rich alginate can bind more cations than M-rich alginate and therefore form a stronger polymer gel matrix. M-rich alginate has fewer binding sites and will, therefore, form a weaker gel when cross-linked. Gels made from M-rich and G-rich alginate will vary in strength and will also bind, such as chelate, different amounts of radionuclide. Variations in the polymer chain length (degree of polymerization, DPn) or in the weight average or number average molecular weight are acceptable, as is known to those skilled in the art.

Thus, the alginate gel of the invention may be based on an M-rich alginate, a G-rich alginate, or a combination thereof. In some embodiments, the alginate gel is based on a G-rich alginate. In preferred embodiments, the average number of G-units in the G-block of the alginate gel is greater than 1. The gel may be a hydrogel, an organogel, or a xerogel. Preferably, the gel is a hydrogel.

The alginate gel of the invention preferably comprises alginate having a molecular weight of 500-350 000, preferably 10 000-250 000, more preferably 25000-150000.

As used herein, the term “alginate gel” refers to any gel formed from an alginate chelating any type of cation. The skilled person is familiar with alginate gels in general, their constitution, and how to form such gels.

The alginate gel of the invention comprises an alginate comprising at least one peptide having a peptide sequence for interacting with a receptor of a cell affected by a proliferative disease, such as a cell attachment peptide, such as a cell-adhesion peptide. As used herein, the term “receptor” means any compound or composition capable of recognising a particular spatial and polar organization of a molecule, i.e., epitopic site. The receptor is typically a cell surface receptor, such as a membrane receptor, such as a transmembrane receptor, and is able to receive, such as bind to, at least one extracellular molecule. Advantageously, the receptor is solely expressed, or over-expressed, by cells affected by a proliferative disease. The interaction of cells with biomaterials is often mediated through cellular receptors that recognise adhesion molecules at material surfaces. The presence in the alginate gel of a peptide having a peptide sequence for interacting with a receptor of a cell affected by a proliferative disease may thus improve cellular adaptability to an alginate gel compared to the corresponding gel without such peptide. It is known from the art that the chemical and physical properties of alginate can be modified by coupling other compounds to the alginate polymer. However, it is known that when a peptide is present on the alginate polymer, the properties of the gel are mainly related to the G-content and the molecular weight of the alginate, rather than the presence and/or amount of a peptide. Further, it has been demonstrated that the amount of peptide does also not affect alginate gelling—which is essentially the same mechanism as is relied upon in the incorporation of radionuclide cations—at least in the range from 3.9×10⁻⁶ to 2×10⁻⁵ mole peptide/g alginate.

The peptide sequence for interacting with a receptor of a cell affected by a proliferative disease is selected from the group of peptide sequences known by the skilled person to be able to interact with, such as bind to, a receptor of a cell affected by a proliferative disease. The receptor is preferably a receptor expressed on the surface of said cell.

The proliferative disease may be malignant or benign. The cell affected by a proliferative disease may be selected from the group comprising or consisting of tumour cells, cancer cells, cells affected by a hyperplastic disease, cells affected by a neoplastic disease. In preferred embodiments, the at least one peptide having a peptide sequence for interacting with a receptor of a cell affected by a proliferative disease is at least one peptide having a peptide sequence for interacting with a receptor of a cancer cell. As used herein, the terms “cancer cell” and “tumour cell” refer to cells that divide at an abnormal, increased rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; and tumours of the nervous system including glioma, glioblastoma multiforme, meningoma, medulloblastoma, schwannoma and ependymoma. The person skilled in the art is knowledgeable about peptide sequences that may be used for interacting with specific types of cells affected by proliferative diseases, in particular cancer cells.

In the present disclosure, the peptides will be referred to using the one-letter abbreviations of the amino acids making up relevant parts of, or the entirety, of their peptide sequence. These abbreviations are well-known to the person skilled in the art. In one embodiment, the peptide sequence is selected from the group comprising integrin-binding peptide sequences, LDL-binding peptide sequences, MMP-2-binding peptide sequences, IL13R2a-binding peptide sequences, VDAC1-binding peptide sequences, NBD-binding peptide sequences, cMYC-binding peptide sequences, CXCR4-binding peptide sequences, MDGI-binding peptide sequences, and combinations thereof. In preferred embodiments, the peptide sequence is selected from the group comprising integrin-binding peptide sequences. As used herein, the term “binding”, when referring to a receptor-binding peptide, may be understood as being able to interact with and/or chemically bind to, said receptor, due to the peptide having a peptide sequence that may interact with said receptor.

Preferably, the at least one peptide is selected from the group comprising or consisting of integrin-binding peptides such as RGD, c(RGDfK), LDL-binding peptides such as TFFYGGSRGKRNNFKTEEY, MMP-2-binding peptides such as MCMPCFTTDHQMARKCDDCCGGKGRGKCYGPQCLCR, IL13R2a-binding peptides such as ACGEMGWGWVRCGGSLCW, VDAC1-binding peptides such as SWTWEKKLETAVNLAWTAGNSNKWTWK, NBD-binding peptides such as TALDWSWLQTE, cMYC-binding peptides such as WPGSGNELKRAFAALRDQI, CXCR4-binding peptides such as RACRFFC, MDGI-binding peptides such as ACGLSGLGVA, EGFR-binding peptides such as YHWYGYTPQNVI, YHWYGYTPENVI, YHWYGYTPQDVI, YHWYGYTPKNVI, YHWYGYTPQKVI, KLARLLT, cyclo (KLARLLT), and NK1-binding peptides such as Substance P (RPKPQQFFGLM). In some embodiments, the at least one peptide is selected from the group of RGD, TFFYGGSRGKRNNFKTEEY, MCMPCFTTDHQMARKCDDCCGGKGRGKCYGPQCLCR, ACGEMGWGWVRCGGSLCW, SWTWEKKLETAVNLAWTAGNSNKWTWK, TALDWSWLQTE, WPGSGNELKRAFAALRDQI, RACRFFC, ACGLSGLGVA, and RPKPQQFFGLM.

In some embodiments, the at least one peptide is selected from Table 1 below, based on the targeted receptor and/or tumour expression.

TABLE 1

| Targeted | | |
|---|---|---|
| receptors | Peptide ligand | Tumor expression |
| Integrin αvβ3 | c(RGDfK) | Glioblastoma, melanoma, breast, prostate |
| | | cancer |
| EGFR | GE11 | Glioblastoma, lung, head and neck cancer |
| | YHWYGYTPQNVI | |
| SSTR2 | Octreotide | NETs, breast, ovarian, cervical cancer |
| | [c[CFwKTC]T(ol) | |
| GnRH-R | [D-Lys⁶]-GnRH-I | Ovarian, breast, endometrial, prostate, lung |
| | pGlu-HWSYkLRPG-NH₂ | cancer |
| Bn receptors | [D-Tyr⁶, β-Ala¹¹, Phe¹³, Nle¹⁴]-Bn(6-14) | Prostate, breast, small cell lung, pancreatic |
| | yQWAV-βAla-HF-Nle-NH₂ | cancer |
| VIP receptors | VIP | Endocrine tumors, colon, breast cancer |
| | HSDAVFTDNYTRLRKQMAVKKYLNSI | |
| | LN-NH₂ | |
| NTSR1 | NT(8-13) | Breast, colon, pancreatic, lung, prostate |
| | RRPYIL | cancer |
| CCK2R | Minigastrin 11 | Gastrointestinal, thyroid, lung, pancreas, |
| | eAYGWMDF-NH₂ | liver cancer |
| MC₁R | NAPamide | Melanoma |
| | Ac-Nle-DHfRWGK-NH₂ | |
| hY₁R | [F⁷, P³⁴]-NPY | Breast cancer, Ewing sarcoma |
| | YPSKPDFPGEDAPAEDLARYYS | |
| | ALRHYINLITRPRY-NH₂ | |

Note:
Exemplary peptide ligands that can be used to address these receptors and the expression of the receptors in human tumors are listed. Abbreviations: Ac, acetyl; Bn, bombesin; c, cyclic; CCK2R, cholecystokinin 2 receptor; e, D-Glu; EGFR, epidermal growth factor receptor; f, D-Phe; GnRH-R, gonadotropin-releasing hormone receptor; hY₁R, human Y₁ receptor; k, D-Lys; MC₁R, melanocortin receptor 1; NET, neuroendocrine tumor; Nle, norleucine; NPY, neuropeptide Y; NT, neurotensin; NTSR1, neurotensin receptor 1; pGlu, pyroglutamic acid; SSTR2, somatostatin receptor type 2; VIP, vasoactive intestinal peptide; w, D-Trp; y, D-Tyr.

The number of peptides having a peptide sequence for interacting with a receptor of a cell affected by a proliferative disease per alginate molecule may vary. In some embodiments, the alginate molecule comprises one peptide having a peptide sequence for interacting with a receptor of a cell affected by a proliferative disease. In preferred embodiments, the alginate molecule comprises at least two peptides having a peptide sequence for interacting with a receptor of a cell affected by a proliferative disease, such as at least three, such as at least five. In some embodiments, the alginate gel of the invention comprises at least two alginate molecules, and at least one of the alginates comprises more of said peptides than another alginate.

In some embodiments, the at least one peptide is selected from the group comprising or consisting of integrin-binding peptides. It is known that integrins play a role in cancer and represent an opportunity to develop therapeutics that can bind specifically to integrins. In specific embodiments, the alginate comprises a peptide having the sequence arginine-glycine-aspartic acid (RGD). The RGD peptide sequence has been shown to bind to integrin αvβ3. It has been shown that alginates comprising a peptide comprising the sequence RGD have the ability to initiate biological interactions between alginate hydrogels and cells, and targeted binding of RGD-peptides to αvβ3 has been used in tumour imaging studies. RGD-alginate, which conveniently is commercially available, can be used to specifically target integrin-expressing tumours. The gel will then be able to bind radionuclides and effectively target an integrin-expressing tumour. The affinity and selectivity for different types of integrin receptors vary among cell types and are dependent on the flanking amino acids of RGD, as well as the conformation and the length of the peptide. Hence, the type of optimal RGD containing peptide sequence and RGD density may vary depending on the cell affected by a proliferative disease to be targeted, as will be understood by the skilled person.

The at least one peptide may be linear. The at least one peptide may be cyclic. Advantageously, certain cyclic peptides have shown increased affinity for cell receptors, e.g., enhanced binding to EGFR and integrin receptors.

The at least one peptide may consist of a peptide sequence for interacting with a receptor of a cell affected by a proliferative disease. Preferably, the peptide comprises a peptide sequence for interacting with a receptor of a cell affected by a proliferative disease. In some embodiments, the peptide comprises a peptide sequence for interacting with a receptor of a cell affected by a proliferative disease and a further peptide sequence, such as a further peptide sequence between the alginate and the peptide sequence for interacting with a receptor of a cell affected by a proliferative disease. The further sequence may e.g. be a “spacer”, i.e. a sequence for ensuring a desired distance between the alginate and the peptide sequence for interacting with a receptor of a cell affected by a proliferative disease, such as for enabling better interaction with the receptor. Non-limiting examples of such further sequences are sequences comprising at least 1-4 glycines, such as GRGDSP, such as GGGGRGDSP.

The at least one peptide having a peptide sequence for interacting with a receptor of a cell affected by a proliferative disease may be linked directly to the alginate, such as via a covalent bond, such as via an amide bond, or the at least one a peptide having a peptide sequence for interacting with a receptor of a cell affected by a proliferative disease may be linked to the alginate via a linking group.

Methods for linking a peptide to an alginate, directly or via a linking group, are well-known in the art; standard chemistry may be used, such as aqueous carbodiimide coupling. Suitable linking groups may readily be determined by the person skilled in the art; for example, a range of linking groups are known from the field of antibody-drug-conjugates. Non-limiting examples of linking groups include poly(ethylene glycol) (PEG) and 2-(maleimidomethyl)-1,3-dioxanes (MD). For the purposes of the invention, the alginate may be prepared by linking the relevant peptide to an alginate, or it may be obtained commercially as a peptide-coupled alginate. For example, the peptide-coupled alginate RGD-alginate is commercially available and sold under the trade name NOVATACH-4GRGDSP.

In some embodiments, the alginate gel of the invention further comprises an alginate that does not comprise a peptide having a peptide sequence for interacting with a receptor of a cell affected by a proliferative disease, such as a non-substituted alginate.

The alginate, and the at least one peptide, may be obtained by any manner known to the skilled person, such as obtained commercially, such as synthesised using any synthetic protocol available to the skilled person, such as enzymatically, synthetically and/or chemically produced.

The alginate gel of the invention further comprises at least one type of divalent cation. The divalent cation is selected such that the alginate and the at least one divalent cation together form an alginate gel. The amounts of cation necessary for gel formation is well-known to the skilled person, and may be determined based on factors such as desired gel strength, type of alginate used (G- or M-rich), and isotonicity of the gelling solution. Concentrations of from 50 to 150 mM are often used. The divalent cation is preferably selected from the group comprising or consisting of Ba²⁺, Sr²⁺, Ca²⁺, Cu²⁺, Zn²⁺, and combinations thereof, more preferably the divalent cation is selected from the group comprising or consisting of Ba²⁺, Sr²⁺, Ca²⁺, and combinations thereof.

The skilled person is knowledgeable about methods for providing alginate gels in different forms. The alginate comprising the at least one peptide and the at least one divalent cation may be gelled e.g. using a diffusion method wherein said alginate is dripped into a solution of said cation (external gelation), in situ gelation using a salt of said cation that is insoluble in water (internal gelation), or by gelation upon cooling, wherein said alginate and said cation are present in solution at high temperature, and the alginate gel is formed upon cooling of the solution.

The alginate gel of the invention further comprises at least one radionuclide cation. As used herein, the term “radionuclide”, which may also be referred to as a radioactive nuclide, radioisotope or radioactive isotope, is an atom that has excess nuclear energy, making it unstable. This excess energy can be used in one of three ways: emitted from the nucleus as gamma radiation; transferred to one of its electrons to release it as a conversion electron; or used to create and emit a new particle (alpha particle or beta particle) from the nucleus. During those processes, the radionuclide is said to undergo radioactive decay. The resulting nuclide is referred to as a daughter or as progeny.

Preferably, the at least one radionuclide is selected from the group comprising or consisting of actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof. More preferably, the at least one radionuclide is selected from actinium-225, actinium-228, bismuth-210, bismuth-211, bismuth-212, lead-211, lead-212, lead-214, radium-223, radium-224, radium-225, strontium-85, strontium-89, thorium-227, thorium-231, thorium-234, yttrium-88, yttrium-90, yttrium-91, and combinations thereof.

In some embodiments, the radionuclide is selected from radium-223 and actinium-225.

In some embodiments, the radionuclide is radium-223. Advantageously, the alginate gel of the invention may also bind the daughter decay nuclides of radium-223, namely polonium-215, lead-211, bismuth-211, thallium-207, and lead-207.

In other embodiments, the radionuclide is actinium-225. As with radium-223, the alginate gel of the invention advantageously also binds the daughter decay nuclides of actinium-225, namely francium 221, and bismuth-213, as well as polonium-213, thallium-209, and lead-209.

As discussed above, alginate-based gels are formed by chelation of cations by alginate through ionic interactions between the cation and lone-pair electrons of oxygen atoms in the hydroxyl groups and in the glycosidic bond. Radionuclide cations can compete for binding to the alginate, such as by replacing some of the original cross-linking cations, thereby themselves becoming part of the gel network. Thus, a radionuclide cation can become a part of the three-dimensional network that is the gel. When a radionuclide cation is chelated by an alginate to form part of an alginate-based gel, the radionuclide is held effectively and firmly in place in the alginate gel through the chelating effect of the radionuclide, yet the alginate structure is sufficiently flexible to absorb recoil energy of an alpha particle emission thereby effectively retaining the newly formed daughter decay nuclide.

In preferred embodiments, the gel is an inhomogeneous gel. As shown in examples 3-7, the radionuclides are effectively not released from such alginate gels.

This manner of retaining an active drug, i.e. a radionuclide, contrasts the frequently used method of encapsulation of drugs in alginate hydrogels. In such hydrogels, the cross-linked alginate polymer molecules are actually holding water in place, and the space occupied by water between the cross-linked polymer chains will act as pores wherein a drug can be encapsulated. The alginate gel will, however, effectively retain molecules above about 50,000 in molecular weight while molecules having lower molecular weight will diffuse out at a rate proportional with their charge and molecular weight. The alginate gel of the invention is based on the principle of chelation, which may allow a significantly more stable incorporation of the active drug than encapsulation in pores as described in the art.

According to the invention, the gel may comprise at least one radionuclide cation having oxidation state 2+ (a divalent cation), oxidation state 3+ (a trivalent cation), or oxidation state 4+ (a tetravalent cation). In some embodiments, the at least one radionuclide cation is at least one divalent radionuclide cation. As the cross-linking cation of alginate-based gels is typically a divalent cation, divalent radionuclide cations can be expected to take the place of these cations with relative ease. Surprisingly, the present inventors have found that when the alginate gel of the invention comprises Ca²⁺, Ba²⁺, and/or Sr²⁺ as the cross-linking cation, the gel is also able to bind the radionuclide lutetium-177, an element having the oxidation state of 3+, and thorium-227, an element having the oxidation state of 4+ The gel may also bind the daughter decay nuclides of thorium-227. As shown in Example 6, approximately 30% of the available thorium-227 is bound to the alginate gel, however 100% of available radium, lead and bismuth nuclides are bound to the alginate gel. Hence, the disclosed alginate gel may be used for purification of thorium-227 by removal of its daughter decay nuclides including radium, lead or bismuth.

Alpha particles, while possessing very high energy, travel only very short distances, usually <100 μm. When at least one radionuclide cation is or comprises an alpha-emitter, a significant number of alpha particles may be lost if the radionuclide cation is located too far into the gel, rather than close to the surface of the gel. Therefore, when the at least one radionuclide cation is or comprises at least one alpha-emitter, at least one radionuclide cation should be located less than 100 μm below the surface of the gel, preferably less than 50 μm, more preferably less than 10 μm below the surface of the gel. Such placement of radionuclide cations may be obtained by introducing the at least one radionuclide cation post-gelling, i.e. by first forming the gel, and then applying the radionuclide cation to the gel, rather than introducing the radionuclide cation during gelling. Thus, radiolabelling of the alginate gel of the invention may advantageously be performed by mixing a solution or a suspension of the radionuclide cation homogeneously with a suspension of the gel, such as in the form of particles, and then separating residual unbound radionuclide cation from the labelled particles, such as by centrifugation or column purification.

The alginate gel of the invention is particularly suitable for delivering radioactive decay in vivo. The gel is biocompatible and biodegradable, and may offer a higher level of physical flexibility and compressibility than what is found e.g. in formulations comprising solid minerals such as that described by ceramics and glass. Further, the presence of a peptide for interacting with tumour cells may contribute to highly selective radiotherapy, ensuring that the alginate gel, and thus the radionuclide, is physically close to the tumour cells. Depending on the form of the alginate gel (e.g., particle vs. block gel), and the type of administration to a subject (e.g., intravenous administration vs. injection directly into a tumour site), the peptide may act as a “targeting unit”, mirroring the targeting units used e.g. in ADC (antibody-drug-conjugate) technology, and/or it may function to ensure that the alginate gel remains in place over time. The physical form of a gel may also contribute to the latter. Further, the stable chelation of the radionuclide and its progeny to the gel ensures efficient radiotherapy with minimal leakage of radioactivity.

In some embodiments, the alginate gel of the invention is in the form of a block gel. In other embodiments, the gel is in the form of a foam, a paste, or an amorphous gel. In yet other embodiments, the gel is in the form of a particle.

In preferred embodiments, the alginate gel of the invention is in the form of at least one particle, such as at least one nanoparticle, such as at least one microparticle, such as at least one rod, such as at least one fiber, such as at least one spray dried and/or freeze-dried particle, preferably as at least one microparticle. Advantageously, particles may easily be incorporated into injectable compositions. Further, the particles may be dry, and thus easily stored.

Thus, in these embodiments, the invention provides an alginate gel particle comprising
an alginate comprising at least one peptide having a peptide sequence for interacting with a receptor of a cell affected by a proliferative disease;
at least one type of divalent cations selected from the group comprising Ba²⁺, Sr²⁺, Ca²⁺, Cu²⁺, Zn²⁺, and combinations thereof; and
at least one radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, 0, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof,
   wherein the alginate chelates the divalent cations and the at least one radionuclide cation.

The embodiments and features described in the context of the alginate gel apply equally to the alginate gel particle.

As used herein, the term “particle” refers to a mass of material, such as a sphere, such as a bead. In some embodiments, the particle is a nanoparticle or a microparticle, preferably a nanoparticle. As used herein, the term “nanoparticle” refers to any particle having a diameter of less than 1000 nm, such as from 1 to 1000 nm. Similarly, the term “nanoparticles” refers to a plurality of particles having an average diameter of between about 1 and 1000 nm. The term “microparticle” refers to any particle having a diameter of less than 1000 μm, such as from 1 to 1000 μm. Similarly, the term “microparticles” refers to a plurality of particles having an average diameter of between about 1 and 1000 μm.

Advantageously, the alginate gel particle has a size of 10-100 000 nm, such as 50-10 000 nm, such as 50 000-80 000 nm, preferably 10-40 000 nm. Reference to the “size” of a particle is a reference to the length of the largest straight dimension of the particle. For example, the size of a perfectly spherical particle is its diameter. The size may e.g. refer to the hydrodynamic radius of the particle characterised by dynamic light scattering.

In some embodiments, the alginate gel particle comprises only one of said peptides. In other embodiments, the particle comprises two or more of said peptides, such as five, such as ten. The number of said peptides may be selected based on the size of the particle. The peptides may be the same or different from each other. The mass ratio of peptide and particle may depend on the molecular weight of the peptide and the diameter of the particle.

In some embodiments, the alginate gel particle comprises one radionuclide cation. In other embodiments, the particle comprises more than one radionuclide cation, such as two, such as three, such as five radionuclide cations, independently selected from the group comprising or consisting of actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof, preferably independently selected from actinium-225, actinium-228, bismuth-210, bismuth-211, bismuth-212, lead-211, lead-212, lead-214, radium-223, radium-224, radium-225, strontium-85, strontium-89, thorium-227, thorium-231, thorium-234, yttrium-88, yttrium-90, yttrium-91, and combinations thereof. It should be noted that in all embodiments, complete control of the number of radionuclide cations per particle cannot be expected; during a radiolabelling process wherein particles are contacted with a solution containing radionuclide cations, a statistical distribution of radionuclide cations per particle will result.

It has been shown that the solutions of an alginate-based gel strongly depend upon the method of preparation. When a gel particle is formed by diffusion of gelling ions into droplets of alginate solution, a non-uniform distribution of polymer in the particle is obtained. This observation can be explained by differences in the diffusion rate of the gelling ions into the particle relative to the diffusion rate of alginate molecules towards the gelling zone. The calcium from the external solution diffuses into the alginate particle. If there are no non-gelling ions present (such as sodium, e.g. from sodium chloride), then the alginate molecules within the gelling particle will move to the outer rim zone of the particle. This creates a localised area of higher alginate concentration than in the middle of the gel particle. This type of gelling is termed “inhomogeneous”, and the resulting gel is referred to as an “inhomogeneous gel”. If, however, a non-gelling ion is present, the non-gelling ion competes for the gelling ion for binding to alginate. The non-gelling ion does not induce gelling but delays the cross-linking action of the gelling ion. In this case, the gel forms with an essentially uniform alginate concentration throughout the alginate particle, termed “homogeneous” gelling. The resulting gel is referred to as a “homogeneous gel”. In both cases the gelling process is almost instantaneous.

More homogeneous particles may be mechanically stronger and have a higher porosity than more inhomogeneous particles. For example, adding sodium chloride together with calcium chloride results in the formation of a more homogeneous particle. Maximum homogeneity is reached with a high molecular weight alginate gelled with high concentrations of both gelling and non-gelling ions. Therefore, while Example 2 shows that homogeneous alginate gel particles in the form of microspheres can be coated with a radionuclide cation, a preferable formulation may utilise inhomogeneous gelling to provide an increased alginate concentration within the outer portions of the alginate gel particle. A higher alginate concentration along the outer section of the gel particle gives strength to the particle as well as providing a larger number of binding sites for, for example, radionuclide cations.

Thus, in preferred embodiments, the alginate gel particle is an inhomogeneous gel particle. In other embodiments, the alginate gel particle is a homogeneous gel particle.

In some embodiments, the alginate gel particle is coated with a poly-cation, such as for giving further strength to the gel if needed and/or for changing the surface charge of the alginate gel from negative to positive charge. Aon-limiting examples of suitable poly-cations are poly-L-lysine. Alternatively, the alginate gel may be coated with the biopolymer chitosan, such as for changing the surface charge to a positive charge. A positive surface charge may e.g. be useful for binding anionic radionuclides.

In another aspect, the invention provides a method for providing at least one alginate gel particle comprising an alginate comprising at least one peptide having a peptide sequence for interacting with a receptor of a cell affected by a proliferative disease and at least one radionuclide cation, wherein the method comprises the steps of
i) providing an aqueous solution of an alginate, wherein the alginate comprises at least one peptide having a peptide sequence for interacting with a receptor of a cell affected by a proliferative disease;
ii) dripping the solution from step i) into an aqueous solution comprising a divalent cation, wherein said divalent cation is selected from Ca²⁺, Sr²⁺, Ba²⁺, Cu²⁺, and Zn²⁺, to form at least one alginate gel particle; and
iii) contacting the alginate gel particle of step ii) with a solution comprising at least one radionuclide cation.

The embodiments and features described in the context of the gel, and in particular the gel particle, above, also apply to the method for providing a gel particle.

The method is particularly suitable for providing microparticles. Advantageously, the method provides gel particles wherein the at least one radionuclide cation is comprised close to the surface of the particle, rather than randomly distributed throughout the entirety of the particle.

The concentration of alginate in the solution of step i) is preferably 0.2-15% (wt/v), more preferably 0.7-5% (wt/v), even more preferably 1.5-2% (wt/v) corrected for dry matter content.

The aqueous solution comprising a divalent cation of step ii) preferably has a total concentration of 10-200 mM, more preferably 50-250 mM, such as 50-100 mM, of said cation. The divalent cation should be a cation that induces gelling, and that is non-toxic or has a tolerable toxicity in humans. In preferred embodiments, the divalent cation of step ii) is selected from the group comprising or consisting of Ca²⁺, Sr²⁺, and Ba²⁺.

The solution of step i) is dripped into the aqueous solution comprising a divalent cation of step ii). The purpose of the dripping may to form particles that are, to a certain degree, spherical. In such instances, the dripping may advantageously be performed using an electrostatic bead generator, which may control the size and size distribution of the particles. However, the particles may alternatively be formed by other means, such as manual or gravity droplet formation, coaxial air-flow, and others techniques known to the skilled person. Several techniques for producing alginate micro- and nanoparticles are known in the art. Another method involves direct spray drying of droplets composed of water-soluble sodium alginate or peptide-coupled alginate and water-soluble calcium chloride. Other technologies can be found in the literature such as oil-in-water emulsification and complexation reactions. The choice of the gelling method or device is not prejudicial to the utility of the invention.

It is also possible to bind radionuclides to non-gel solid particles of alginate.

For reference, further examples of procedures for preparing alginate gels suitable for use in humans can be found e.g. in ASTM Guideline F2315.

It is an advantage of the disclosed method that the at least one gel particle resulting from step ii) can be pre-formed and/or stored, and step iii) performed at a later time. For instance, the gel particles of step ii) may be stored, such as in the solution of step ii), until needed. Alternatively, the gel particles of step ii) may be subjected to a spray drying or freeze-drying process. It has been shown that sterile alginate microspheres stored in water or NaCl solutions are stable for years, especially if there is a little calcium in the solution. Example 9 further illustrates the stability of alginate gel particles.

In some embodiments, step iii) is performed in a buffer solution to maintain a pH of 3 to 8. The use of such buffer may contribute to maintaining the structural integrity of the gel particle. The duration of step iii) is typically 20-180 minutes, such as 30-120 minutes. If freeze-dried gel particles are used, the radionuclide solution volume should be adjusted to allow for rehydration of the particles, as known to the skilled person.

The method disclosed above utilises the concept of external gelation. It has been found by the inventors that for the preparation of the gel particles of the invention, the disclosed method is more advantageous compared to performing the gelling with the radionuclide cation co-mixed with the cation of step ii). The latter results in incorporation of the radionuclide cation within the gel particle, rather than close to the surface as in the claimed method. As discussed above, alpha particles, while possessing very high energy, travel only very short distances, usually <100 μm. Therefore, incorporating an alpha particle emitting radionuclide cation within a particle having a size of e.g. >100 μm would result in loss of a significant number of alpha particles due to absorption of energy within the particle itself. Thus, the method of the invention is particularly useful when particles comprising an alpha emitter are to be prepared.

The alginate gel particles of the invention may be made either by homogeneous or inhomogeneous gelling procedures, i.e. with alginate solutions and gelling ion solutions containing an amount of non-gelling ion, such as NaCl, typically up to 0.9% in concentration, for homogeneous gelling, or not containing addition of salt to create isotonicity. The gelling solution may be made isotonic without NaCl by the use of mannitol. Hence, in some embodiments, the aqueous solution comprising a divalent cation of step ii) further comprises NaCl, such as for homogenous gelling. In other embodiments, when inhomogeneous gelling is desired, said solution does not comprise substantial amounts of NaCl.

Advantageously, the method may be performed aseptically by using sterile alginate solution, sterile cation gelling solution, and sterilised equipment used to generate particles may be sterilised by appropriate means. For example, sterile alginate gel microspheres can be produced by dissolving sterile, lyophilised sodium alginate in sterile water or other appropriate dilutant. Equipment for the production of microspheres, such as the NISCO VAR1 electrostatic bead generator, can be sterilised by autoclaving and ethanol disinfection. The gelling bath solution can be sterilised by filter sterilisation. The entire production of alginate gel microspheres can be performed by placing the equipment in a laminar air flow (LAF) sterile bench.

Further, introduction of radionuclide cation into pre-formed, sterile alginate gel microspheres can be performed with a sterile solution of appropriate radionuclide cation.

Following incubation to coat the gel microspheres, washing can be performed by the use of sterile solutions. Radionuclide-coated, sterile alginate gel microspheres can be stored in sterile containers.

The alginate gel disclosed herein may be present as an active ingredient in a desired dosage unit formulation, such as a pharmaceutically acceptable composition containing a conventional pharmaceutically acceptable carrier. As used herein, the term “pharmaceutically acceptable” means that compound must be physiologically acceptable to the recipient as well as, if part of a composition, compatible with other ingredients of the composition. The term “composition” refers to a mixture, in any formulation, of one or more compounds according to the invention with one or more additional chemical component.

Thus, in another aspect, the invention relates to a composition comprising an alginate gel comprising
an alginate comprising at least one peptide having a peptide sequence for interacting with a receptor of a cell affected by a proliferative disease;
at least one type of divalent cations selected from the group comprising Ba²⁺, Sr²⁺, Ca²⁺, Cu²⁺, Zn²⁺, and combinations thereof; and
at least one radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof, wherein the alginate chelates the divalent cations and the at least one radionuclide cation together with at least one pharmaceutically acceptable carrier, diluent, and/or excipient.

In preferred embodiments, the gel is in the form of a particle, such that the invention relates to a composition comprising
an alginate gel particle comprising
   an alginate gel comprising
   an alginate comprising at least one peptide having a peptide sequence for interacting with a receptor of a cell affected by a proliferative disease;
at least one type of divalent cations selected from the group comprising Ba²⁺, Sr²⁺, Ca²⁺, Cu²⁺, Zn²⁺, and combinations thereof; and
   at least one radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof,
   wherein the alginate chelates the divalent cations and the at least one radionuclide cation
      together with at least one pharmaceutically acceptable carrier, diluent, and/or excipient.

The embodiments and features described in the context of the gel, and in particular the gel particle, above, also apply to the composition of the invention.

The composition may be considered to be a pharmaceutical composition, as it comprises an active agent, i.e. the gel, combined with at least one pharmaceutically acceptable carrier, diluent, and/or excipient, making the composition especially suitable for therapeutic use.

The composition preferably comprises a multitude of the alginate gel particles of the invention. The particles can be the same of different, i.e. with regards to type and number of radionuclide cations and/or peptides. In some embodiments, the composition is a particle suspension comprising monodisperse or polydisperse particles labelled with a radionuclide cation.

The composition may further include one or more of any conventional, pharmaceutically acceptable excipients and/or carriers, e.g. solvents, fillers, diluents, binders, lubricants, glidants, viscosity modifiers, surfactants, dispersing agents, disintegration agents, emulsifying agents, wetting agents, suspending agents, thickeners, buffers, pH modifiers, absorption-delaying agents, stabilisers, antioxidants, preservatives, antimicrobial agents, antibacterial agents, antifungal agents, chelating agents, adjuvants, sweeteners, aromas, and colouring agents. Conventional formulation techniques known in the art, e.g., conventional mixing, dissolving, suspending, granulating, levigating, emulsifying, encapsulating, or entrapping, may be used to formulate the composition.

In some embodiments, the composition is formulated for a particular method of administration to a subject.

The amount of gel according to the invention present in the composition can vary. In some embodiments, the amount of gel according to the invention present in the composition is 0.1-50% by weight, such as 1-30%, such as 50-20%. In other embodiments, the amount of the gel according to the invention present in the composition is 30-70% by weight, such as 40-60%. In yet other embodiments, the amount of the gel according to the invention present in the composition is 50-100% by weight, such as 50-70%, such as 50-80%, such as 60-98%, such as 70-95%.

The composition may also comprise alginate gel, such as alginate gel particles, that do not comprise a radionuclide cation. Such alginate gel may be the same as the alginate gel of the invention except for the absence of the radionuclide cation, or it may be different. The ratio between alginate gel that comprises a radionuclide cation and alginate gel that does not comprise a radionuclide cation may vary. In preferred embodiments, at least 90% of the alginate gel comprises a radionuclide cation. The activity per mg gel may e.g. range from 0.1 kBq/mg to 100 kBq/mg, but will depend on e.g. the choice of radionuclide.

Further, the composition is substantially free of contaminants or impurities. In some embodiments, the level of contaminants or impurities other than residual solvent in the composition is below about 5% relative to the combined weight of the gel according to the invention and the intended other ingredients. In certain embodiments, the level of contaminants or impurities other than residual solvent in the composition is no more than about 2% or 1% relative to the combined weight of the gel according to the invention and the intended other ingredients.

In certain embodiments, the gel or composition according to the invention is sterile. The gel may be prepared antiseptically as outlined above. Sterilisation can be achieved by any suitable method, including but not limited to by applying heat, chemicals, irradiation, high pressure, filtration, or combinations thereof.

In another aspect, the invention relates to a kit comprising,
in a first container,
an alginate gel comprising
   an alginate comprising
      at least one peptide having a peptide sequence for interacting with a receptor of a cell affected by a proliferative disease; and
      at least one type of divalent cations selected from the group comprising Ba²⁺, Sr²⁺, Ca²⁺, Cu²⁺, Zn²⁺, and combinations thereof; and
   optionally, a suitable solvent, and
in a second container,
   at least one radionuclide cation, and
   a suitable solvent.

In some embodiments, the gel is in the form of a gel particle. In some embodiments, all contents of the first and the second containers are sterile. In some embodiments, the solvent or solvents are pharmaceutically acceptable.

The concentrations of the contents of the first and second containers may be selected based on e.g. the desired level of radiolabelling per unit, such as particle, of gel. The concentrations of the components of the first and second containers may be selected based on the skilled person's knowledge of pharmaceutically acceptable compositions. In some embodiments, the gel is freeze-dried, and the first container does not contain a solvent.

The skilled person will appreciate that the first and/or the second container may further comprise further components, such as at least one carrier, diluent, and/or excipient.

Internal gelation can be achieved by restricting the amount of gelling ions available for complexation with the alginate, for example mixing alginate solutions with CaCO₃, which has low solubility at neutral pH, but higher solubility at lower pH, and a slowly hydrolysing substance, such as glucono-δ-lactone (GDL). As the GDL hydrolyses and the pH then drops, the Ca-salt dissolves, making the Ca²⁺ ions available for gelling. Another method for making alginate gels by internal gelling is by mixing the alginate solution with a mixture of quickly and/or slowly dissolving gelling ion salts, for example CaCl₂) and CaSO₄. Internal gelation can also be carried out by encapsulating the gelling ions in liposomes and mixing the liposomes with an alginate solution. The liposomes can then be destabilised, i.e. by thermoactivation, and subsequent release of the gelling ions causes gelation.

Delayed gelation systems, as disclosed in e.g. WO2006044342A2 and WO2009032158, represents a useful way to create alginate-based gels by internal gelation. Its composition is simple and requires no extra additives; the only substituents needed are a soluble alginate and an insoluble alginate, e.g. sodium and calcium alginate. The system will gel after a controllable time, as the saturated calcium alginate donates calcium ions to the dissolved sodium alginate. As the delayed gelation system facilitates the production of near-homogenous gels at a controlled rate without any need for pH changes or addition of inorganic salts, this system poses as an alternative to alginate gel production by diffusion/dialysis or by the use of combination of calcium salts and acidic agents. The delayed gelation alginate system has been used e.g. as a tissue bulking agent in left ventricle cardiac regeneration after cardiac infarct.

The incorporation of one or more radionuclide cations as disclosed above into a delayed gelation alginate system, wherein the alginate comprises at least one peptide having a peptide sequence for interacting with a receptor of a cell affected by a proliferative disease, represents a useful tool for internal radiotherapy, allowing direct injection into a tumour or into a tumour resection site. As the gelling reaction is delayed, there is sufficient time for injection or implantation of the gel solution prior to the onset of gelation. Gelation takes place in situ, resulting in a radioactive gel at the desired site.

Thus, in another aspect, the invention relates to a kit comprising,
in a first container,
   a water-soluble alginate,
      wherein the alginate comprises at least one peptide having a peptide sequence for interacting with a receptor of a cell affected by a proliferative disease,
   water, and
   optionally, another suitable solvent;
in a second container,
   particles that are insoluble in water, said particles comprising an alginate and at least one type of divalent cation selected from the group comprising Ba²⁺, Sr²⁺, Ca²⁺, Cu²⁺, Zn²⁺, and
   water; and
in a third container
   at least one radionuclide cation, and
   a suitable solvent.

The soluble alginate may be selected from sodium alginate, magnesium alginate, and/or potassium alginate, wherein the alginate has a molecular weight of 500-350 000, preferably 10 000-250 000, more preferably 25 000-150 000.

When in use, the contents of the third container may be mixed with the contents of the second container, and the mixture added to the contents of the first container in order to form a delayed gelling system for forming a radioactive gel.

The concentrations of the contents of the first, second, and third containers may be selected based on e.g. the desired level of radiolabelling per unit of gel. The concentrations of the components of the first, second, and third containers may be selected based on the skilled person's knowledge of pharmaceutically acceptable compositions.

The skilled person will appreciate that the first, second, and/or third container may further comprise further components, such as at least one carrier, diluent, and/or excipient.

In another aspect, the invention relates to
an alginate gel comprising
   an alginate comprising at least one peptide having a peptide sequence for interacting with a receptor of a cell affected by a proliferative disease;
   at least one type of divalent cations selected from the group comprising Ba²⁺, Sr²⁺, Ca²⁺, Cu²⁺, Zn²⁺, and combinations thereof; and
   at least one radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof,
   wherein the alginate chelates the divalent cations and the at least one radionuclide cation
or a composition comprising
an alginate gel comprising
   an alginate comprising at least one peptide having a peptide sequence for interacting with a receptor of a cell affected by a proliferative disease;
   at least one type of divalent cations selected from the group comprising Ba²⁺, Sr²⁺, Ca²⁺, Cu²⁺, Zn²⁺, and combinations thereof; and
   at least one radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof,
   wherein the alginate chelates the divalent cations and the at least one radionuclide cation together with at least one pharmaceutically acceptable carrier, diluent,
   and/or excipient,
for use as a medicament.

The gel is defined as disclosed herein. In preferred embodiments, the gel is in the form of a particle.

The alginate gel of the invention, and the composition comprising said gel, may be used therapeutically, such for delivery of radioactive decay to one or more particular sites in vivo, such as a particular cell, tissue, organ, etc, with the advantages outlined above for the gel.

For the various aspects of the invention as described herein that relate to use as a medicament and/or treatment of disease, particularly relating to cells infected by a proliferative disease, the cells may in all embodiments reside at a single site in the body, for example in the case of a localised solid tumour, or may reside at a plurality of sites, for example in the case of a distributed or metastasised cancerous disease.

Due to the cytotoxic effects of radioactive decay, the gel and the composition of the invention may be particularly useful against proliferative diseases. Thus, in a further aspect, the invention relates to
an alginate gel comprising
   an alginate comprising at least one peptide having a peptide sequence for interacting with a receptor of a cell affected by a proliferative disease;
   at least one type of divalent cations selected from the group comprising Ba²⁺, Sr²⁺, Ca²⁺, Cu²⁺, Zn²⁺, and combinations thereof; and
   at least one radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof,
   wherein the alginate chelates the divalent cations and the at least one radionuclide cation
or a composition comprising
an alginate gel comprising
   an alginate comprising at least one peptide having a peptide sequence for interacting with a receptor of a cell affected by a proliferative disease;
   at least one type of divalent cations selected from the group comprising Ba²⁺, Sr²⁺, Ca²⁺, Cu²⁺, Zn²⁺, and combinations thereof; and
   at least one radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof,
   wherein the alginate chelates the divalent cations and the at least one radionuclide cation
   together with at least one pharmaceutically acceptable carrier, diluent, and/or excipient,
for use in a method for the treatment of a proliferative disease.

The gel is defined as disclosed herein. In preferred embodiments, the gel is in the form of a particle.

The terms “treating” and “treatment” and “therapy” (and grammatical variations thereof) are used herein interchangeably, and refer to 1) inhibiting the disease; for example, inhibiting a disease, condition or disorder in a subject who is experiencing or displaying the pathology or symptomatology of the disease, condition or disorder, including prevention of disease (i.e. prophylactic treatment, arresting further development of the pathology and/or symptomatology), or 2) alleviating the symptoms of the disease, or 3) ameliorating the disease; for example, ameliorating a disease, condition or disorder in an subject who is experiencing or displaying the pathology or symptomatology of the disease, condition or disorder (i.e., reversing the pathology and/or symptomatology). The terms may relate to the use and/or administration of medicaments, active pharmaceutical ingredients (API), and/or pharmaceutical grade supplements.

As used herein, the terms “administer”, “administration”, and “administering” refer to (1) providing, giving, dosing and/or prescribing by either a health practitioner or their authorised agent or under their direction, or by self-administration, a formulation, preparation or composition according to the present disclosure, and (2) putting into, taking or consuming by the subject themselves, a formulation, preparation or composition according to the present disclosure. In preferred embodiments, the use comprises administration of the alginate gel or composition by a health practitioner or authorised agent.

As used herein, “subject” means any human or non-human animal selected for treatment or therapy, and encompasses, and may be limited to, “patient”. None of the terms should be construed as requiring the supervision (constant or otherwise) of a medical professional (e.g., physician, nurse, nurse practitioner, physician's assistant, orderly, clinical research associate, etc.) or a scientific researcher.

The subject is preferably a human subject. The subject may be male or female. In some embodiments, the subject is an adult (i.e. 18 years of age or older). In certain embodiments, the subject is geriatric. In certain embodiments, the subject is not geriatric. The subject is preferably a subject that has been diagnosed with a proliferative disease, such as a cancer.

The diseased tissue to be targeted may be at a soft tissue site, at a calcified tissue site or a plurality of sites which may all be in soft tissue, all in calcified tissue or may include at least one soft tissue site and/or at least one calcified tissue site. In one embodiment, at least one soft tissue site is targeted. The sites of targeting and the sites of origin of the disease may be the same, but alternatively may be different. Where more than one site is involved this may include the site of origin or may be a plurality of secondary sites. The term “soft tissue” is used herein to indicate tissues which do not have a “hard” mineralised matrix. In particular, soft tissues as used herein may be any tissues that are not skeletal tissues. Correspondingly, “soft tissue disease” as used herein indicates a disease occurring in a “soft tissue” as used herein. The invention is particularly suitable for the treatment of cancers and “soft tissue disease” thus encompasses carcinomas, sarcomas, myelomas, leukaemias, lymphomas and mixed type cancers occurring in any “soft” (i.e. non-mineralised) tissue, as well as other non-cancerous diseases of such tissue. Cancerous “soft tissue disease” includes solid tumours occurring in soft tissues as well as metastatic and micro-metastatic tumours. Indeed, the soft tissue disease may comprise a primary solid tumour of soft tissue and at least one metastatic tumour of soft tissue in the same patient. Alternatively, the “soft tissue disease” may consist of only a solid tumour or only metastases with the primary tumour being a skeletal disease.

In a specific embodiment, the method of treatment, or use in a method for the treatment, comprises treatment with the alginate gel of the invention coordinated with surgery, such as cancer surgery. For instance, as part of a surgical procedure wherein cancerous tissue is or has been removed, the alginate gel is administered into the area of the cancer, such as into a cavity where cancerous tissue has been removed. Preferably, such administration and treatment is performed as part of the surgical procedure, concurrently with the surgery or shortly after the surgery. The administered radionuclides will accordingly deliver radioactive decay to the particular site where cancerous tissue has been removed, and the administered radionuclide cation and/or its daughters can provide their toxic effect to tissue and cells in the treatment area.

When the gel or composition of the invention is used as a medicament and/or in a method of treatment according to the invention, the at least one peptide having a peptide sequence for interacting with a receptor of a cell affected by a proliferative disease is selected such that they are suitable for interacting with, such as binding to, a receptor of a cell affected by the specific proliferative disease to be treated, such as based on Table 1

In some embodiments, said proliferative disease is a cancer, a non-cancerous tumour, a neoplastic disease, or a hyperplastic disease. In some embodiments, said proliferative disease is selected from the group of atherosclerosis, rheumatoid arthritis, psoriasis, idiopathic pulmonary fibrosis, scleroderma, and cirrhosis of the liver.

In specific embodiments, said proliferative disease is a cancer. As used herein, the terms “cancer” and “tumour” refer to any neoplastic growth in a subject, including an initial tumour and any metastases. The cancer can be of the liquid or solid tumour type. Liquid tumours include tumours of haematological origin, including, e.g., myelomas (e.g., multiple myeloma), leukaemia (e.g., Waldenstrom's syndrome, chronic lymphocytic leukaemia, other leukaemias), and lymphomas (e.g., B-cell lymphomas, non-Hodgkin's lymphoma). Solid tumours can originate in organs and include, but are not limited to, cancers of the lungs, brain, breasts, prostate, ovaries, colon, kidneys and liver.

In some embodiments, the cancer is selected from the list comprising or consisting of lung cancer, pancreatic cancer, colorectal cancer; liver cancer, glioma, renal cancer, non-Hodgkin's lymphoma, neuroblastoma, CNS metastases, peritoneal cancer, follicular lymphoma, colorectal cancer, small cell lung cancer, carcinoma, sarcoma, myeloma, leukaemia, lymphoma, prostate cancer or mixed type cancer.

In specific embodiments, the cancer is a metastatic cancer. Treatment of metastatic cancers is notoriously difficult using conventional anticancer therapies, but the targeted alginate vehicles of the invention represent a promising line of treatment for such cancer.

The gel or composition for use as a medicament and/or in a method of treatment according to the invention will be administered to a subject in a therapeutically effective dose. The term “therapeutically effective dose” as used herein means the amount of gel according to the invention which is effective for producing the desired therapeutic effect in a subject at a reasonable benefit/risk ratio applicable to any treatment. The therapeutically effective dosage amount may vary depending upon the route of administration and dosage form. Further, dosages may depend on the particle to be used, the type of radioactive decay of the radionuclide cation and/or its daughters, the stage of the condition, age and weight of the subject, etc. and may be routinely determined by the skilled practitioner according to principles well known in the art.

In some embodiments, the amount of radionuclide cation used per patient dosage may be in the range of from 1 kBq to 10 GBq, preferably 1 kBq to 100 MBq, more preferably 10 kBq MBq to 25 MBq, even more preferably in the range of from 10 kBq to 10 MBq. The dosage and the maximum dosage may be determined by the person skilled in the art based on common general knowledge about suitable dosages and maximum dosages. It is accepted in the art that a realistic and conservative estimate of the toxic side effects of daughter isotopes must be adopted.

In some embodiments, the particles are administered at a dosage of 10 Bq/kg-100 MBq/kg bodyweight. Correspondingly, a single dosage unit may comprise around any of these ranges multiplied by a suitable bodyweight, such as 30 to 150 Kg, preferably 50 to 100 kg. The dosage may depend on the choice of radionuclide, its half-life and/or the specific progeny. The dosage, the gel and the administration route may be such that the dosage of progeny generated in vivo is less than 300 kBq/kg, such as less than 200 kBq/kg, preferably less than 150 kBq/kg, such as less than 100 kBq/kg.

The alginate gel or composition for use as a medicament and/or in a method of treatment according to the invention may be administered locally or systemically. The gel or composition according to the invention may be administered by an administration route selected from the group comprising or consisting of intratumour, intrathecal, intravenous, and intraarterial, such as embolic therapy. The choice of administration route may be selected based on the form of the gel. For intravenous administration, the gel should be in the form of nanoparticles.

The therapeutically effective dose of the alginate gel or composition according to the invention can be administered in a single dose or in divided doses. The alginate gel or composition according to the invention can be administered once, twice or more times a day, once every two days, once every three days, twice a week or once a week, or as deemed appropriate by a medical professional. In certain embodiments, the alginate gel or composition according to the invention is administered once daily. In other embodiments, the alginate gel or composition according to the invention is administered twice daily. In some embodiments, the dosage regimen is predetermined and the same for the entire patient group. In other embodiments, the dosage and the frequency of administration of treatment with the alginate gel or composition according to the invention is determined by a medical professional, based on factors including, but not limited to, the stage of the disease, the severity of symptoms, the route of administration, the age, body weight, general health, gender and/or diet of the subject, and/or the response of the subject to the treatment.

In some embodiments, the therapeutically effective dose is administered at regular intervals. In other embodiments, the dose is administered when needed or sporadically. The alginate gel or composition according to the invention may be administered by a medical professional. The alginate gel or composition according to the invention may, depending on factors such as formulation and route of administration, be administered with food or without food. In some embodiments, the alginate gel or composition according to the invention is administered at specific times of day.

In some embodiments, the alginate gel or composition is administered orally. In some embodiments, the alginate gel or composition is administered with a meal or before a meal. In some embodiments, the alginate gel or composition according to the invention is administered intravenously. In these embodiments, water is a particularly useful excipient. Saline solutions and aqueous dextrose and glycerol solutions can also be employed as liquid excipients, particularly for injectable solutions.

Preferred unit dosage formulations are those containing a therapeutically effective dose, as hereinbefore recited, or an appropriate fraction thereof, of a gel according to the invention. A composition of the invention may be presented in unit dosage form as a single dose wherein all active and inactive ingredients are combined in a suitable system and components do not need to be mixed before administration. Alternatively, a composition may be presented as a kit, such as a kit disclosed above, and may contain instructions for storing, preparing, administering and/or using the composition.

In some embodiments, the duration of the use of the alginate gel or composition for use as a medicament and/or in a method of treatment according to the invention is determined by the observed effect of the treatment, such as by reduction of and/or amount of target antigen expression. In some embodiments, treatment is sustained until no further improvement can be expected. In certain embodiments, the duration of the treatment with the alginate gel or composition according to the invention is at least two weeks, at least one month, at least three months, such as three months, six months, nine months, a year, three years, five years. In other embodiments, the duration is determined by a medical professional, based on factors including but not limited to the nature and severity of the symptoms, the route of administration, the age, body weight, general health, gender and/or diet of the subject, and/or the response of the subject to the treatment.

In certain embodiments, the alginate gel or composition according to the invention is administered alone. In other embodiments, the alginate gel or composition according to the invention is administered in combination with one or more other therapeutic agents. Said one or more other therapeutic agents may be known to have an effect against a proliferative disease, such as cancer, and/or may have an additive or synergistic mechanism of action on treatment of said proliferative disease, such as a cancer, together with the alginate gel or composition of the invention. In some embodiments, the alginate gel or composition according to the invention is administered as part of a combination therapy. Combination therapies comprising an alginate gel or composition according to the invention may refer to compositions that comprise the alginate gel or composition according to the invention in combination with one or more therapeutic agents, and/or co-administration of the alginate gel or composition according to the invention with one or more therapeutic agents wherein the alginate gel or composition according to the invention and the other therapeutic agent or agents have not been formulated in the same composition. When using separate formulations, the alginate gel or composition according to the invention may be administered simultaneously, intermittent, staggered, prior to, subsequent to, or combinations of these, with the administration of another therapeutic agent.

The embodiments and features described in the context of one aspect, e.g. for the aspect directed to the alginate gel or the composition, also apply to the other aspects of the invention, such as the use thereof as a medicament or in a method of treatment.

In a further aspect, the invention provides a method of treatment, the method comprising the step of administering an effective amount of a gel or composition of the invention, to a subject in need thereof.

In a further aspect, the invention provides a method of treatment of a proliferative disease, the method comprising the step of administering an effective amount of a gel or composition of the invention, to a subject in need thereof.

In a further aspect, the invention provides the use of a gel or composition of the invention as a medicament.

In a further aspect, the invention provides the use of a gel or composition of the invention for treatment of a proliferative disease.

In a further aspect, the invention provides a kit comprising,
in a first container,
an alginate gel comprising
   an alginate comprising
      at least one peptide having a peptide sequence for interacting with a receptor of a cell affected by a proliferative disease; and
      at least one type of divalent cations selected from the group comprising Ba²⁺, Sr²⁺, Ca²⁺, Cu²⁺, Zn²⁺, and combinations thereof; and
optionally, a suitable solvent; and
in a second container,
   at least one radionuclide cation, and
   a suitable solvent
for use as a medicament.

In a further aspect, the invention provides a kit comprising,
in a first container,
an alginate gel comprising
   an alginate comprising
      at least one peptide having a peptide sequence for interacting with a receptor of a cell affected by a proliferative disease; and
      at least one type of divalent cations selected from the group comprising Ba²⁺, Sr²⁺, Ca²⁺, Cu²⁺, Zn²⁺, and combinations thereof; and optionally, a suitable solvent; and
in a second container,
   at least one radionuclide cation, and
   a suitable solvent
for use in the treatment of a proliferative disease.

In a further aspect, the invention provides a kit comprising,
in a first container,
   a water-soluble alginate,
      wherein the alginate comprises at least one peptide having a peptide sequence for interacting with a receptor of a cell affected by a proliferative disease,
   water, and
   optionally, another suitable solvent;
in a second container,
   particles that are insoluble in water, said particles comprising an alginate and at least one type of divalent cation selected from the group comprising Ba²⁺, Sr²⁺, Ca²⁺, Cu²⁺, Zn²⁺, and combinations thereof, and water; and
in a third container
   at least one radionuclide cation, and
   a suitable solvent
for use as a medicament.

In a further aspect, the invention provides a kit comprising,
in a first container,
   a water-soluble alginate,
      wherein the alginate comprises at least one peptide having a peptide sequence for interacting with a receptor of a cell affected by a proliferative disease,
   water, and
   optionally, another suitable solvent;
in a second container,
   particles that are insoluble in water, said particles comprising an alginate and at least one type of divalent cation selected from the group comprising Ba²⁺, Sr²⁺, Ca²⁺, Cu²⁺, Zn²⁺, and combinations thereof, and
   water; and
in a third container
   at least one radionuclide cation, and
   a suitable solvent
for use in the treatment of a proliferative disease.

The invention shall not be limited to the shown embodiments and examples. While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is to be understood that various alternatives to the embodiments described herein can be employed in practicing the disclosure.

It is to be understood that every embodiment of the disclosure can optionally be combined with any one or more of the other embodiments described herein.

It is to be understood that each component, compound, particle, or parameter disclosed herein is to be interpreted as being disclosed for use alone or in combination with one or more of each and every other component, compound, or parameter disclosed herein. It is further to be understood that each amount/value or range of amounts/values for each component, compound, or parameter disclosed herein is to be interpreted as also being disclosed in combination with each amount/value or range of amounts/values disclosed for any other component(s), compound(s), or parameter(s) disclosed herein, and that any combination of amounts/values or ranges of amounts/values for two or more component(s), compound(s), or parameter(s) disclosed herein are thus also disclosed in combination with each other for the purposes of this description. Any and all features described herein, and combinations of such features, are included within the scope of the present invention provided that the features are not mutually inconsistent.

It is to be understood that each lower limit of each range disclosed herein is to be interpreted as disclosed in combination with each upper limit of each range disclosed herein for the same component, compound, or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, or parameter disclosed in the description or an example is to be interpreted as a disclosure of either a lower or an upper limit of a range and thus can be combined with any other lower or upper limit or a range or specific amount/value for the same component, compound, or parameter disclosed elsewhere in the application to form a range for that component, compound, or parameter.

### EXAMPLES

### Example 1: Preparation of Alginate Gel Particles

Sodium alginate (PRONOVA LVG) was first dissolved in distilled water to give a 2% solution. Alginate gel particles in the form of microspheres of an average diameter of 150 μm were formed using a NISCO electrostatic bead generator (model VAR1) having a 0.1 mm (for 150 μm bead diameter) nozzle, a flow rate of from 2-5 ml/hr, an electrostatic potential between the nozzle and the gelling bath of 6500 KV and a distance from the nozzle to the surface of the gelling bath of approximately 1.5 cm. The gelling bath contained 50 mM aqueous solution of CaCl₂). FIG. 2 shows a photomicrograph of the resulting alginate gel microspheres, taken by a digital camera mounted on the photo tube of a Nikon microscope.

In the following Examples both M-rich (identified as LVM) and G-rich (identified as LVG) alginate was used. In some of the examples, sodium alginate (PRONOVA LVG or PRONOVA SLG) was used. The examples using sodium alginate can also be considered as representative examples for peptide-coupled alginate since the conjugation of a peptide will typically not affect the gelling capability of the alginate. The commercially available NOVATACH peptide-coupled alginate contains from 0.8 to 0.4% peptide (either GRGDSP or GGGGRGDSP). This low peptide: alginate ratio will not affect the functionality of the alginate, including its ability to chelate radionuclide cations. Other examples presented herein have used NOVATACH peptide-coupled alginate where appropriate.

### Example 2: Binding of Ra-223 and Daughter Nuclides to Calcium Crosslinked, Homogeneous Gelled Alginate Microspheres—Examples of Divalent Radionuclide Cations

In this Example, binding of Ra-223 and daughter nuclides to calcium crosslinked, homogeneous gelled alginate microspheres, and the stability of said binding, was examined.

A known amount of pre-formed alginate gel microspheres was washed successively to remove excess gelling ion, using 0.9% solution of NaCl. Radium-223, present as ²²³RaCl₂ in 8N HNO₃, was diluted in an aliquot of phosphate buffered saline (PBS). The radioactive content (measured as Bq/ml) of this solution was determined by a dose calibrator (Capintec). About 600 mg of alginate microspheres (weight) was then mixed with ca. 300 kBq/mL of radioactivity. For binding of radium isotopes, room temperature conditions, intermittent shaking, and 60-minute incubation conditions were found to be sufficient to bind all available radium and the decay daughters of radium-223, lead-211 and bismuth 211. The Ra-223-coated alginate gel microspheres were then washed 3 times using PBS. The radioactivity associated with the alginate microspheres was determined by gamma spectroscopy using high performance germanium detector (HPGe) which can simultaneously detect radium-223 and daughter radionuclides lead-2111 and bismuth-211. The radioactive nuclide-coated alginate gel microspheres were stored in PBS solution.

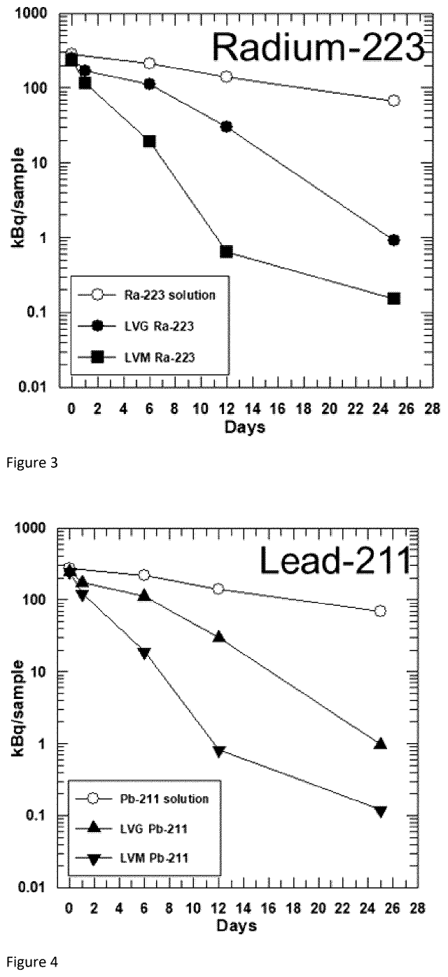

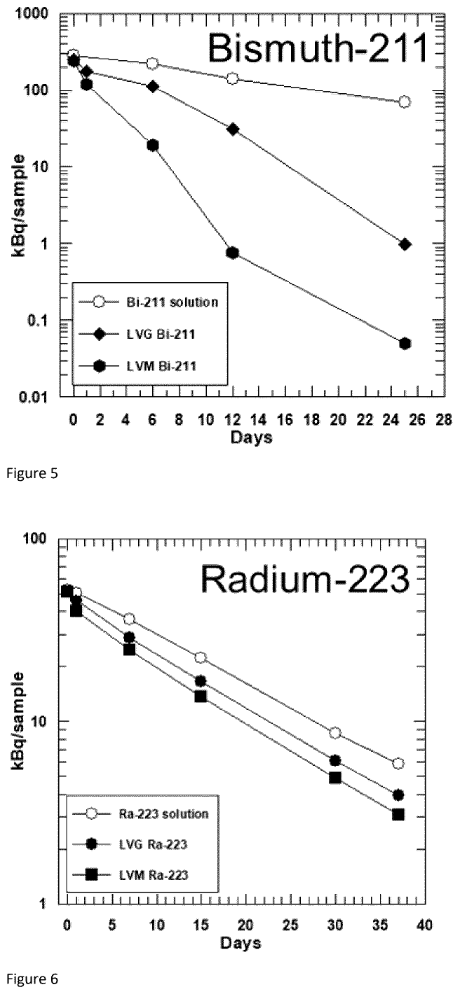

The radioactivity from bound radium-223 and its decay daughters in samples containing a similar weight (500 mg) of alginate microspheres was then determined using a HPGe to measure on samples containing a similar weight (500 mg) of alginate microspheres. FIGS. 3-5 show the results from these measurements. In the Figures, “Ra-223 solution” indicates the sample not containing alginate microspheres, “LVG” indicates G-rich alginate, and “LVM” indicates M-rich alginate. The data, kBq/sample versus the day of analysis, was plotted using GraFit software. The slope of the curve represents the radioactive decay of the radionuclide.

As can be seen from the figures, while the alginate microspheres bind radium-223 and decay daughters, there appears to be a certain release of the radionuclides from the gel network over time as shown by the steeper slope of the bound radioactivity curves relative to the curve for the radionuclide in solution during time. In addition, it appears that the M-rich alginate (LVM) is further reduced in the ability to retain the daughter decay radionuclides. This indicates a weaker binding of radium-223 and decay daughters to LVM alginate but, nonetheless, radionuclide binding does occur.

In this Example, homogeneous gelation of alginate was used to form the microspheres, i.e. there were non-gelling ions present in the form of Na⁺ from NaCl. Examples 3 and 4 below show the effect of producing alginate microspheres by the inhomogeneous gelling technique, i.e. no Na⁺ present.

In this Example, radium-223 was used, however, similar conditions and results may be obtained using other radionuclides.

### Example 3: Binding of Radionuclides and Decay Daughter Nuclides to Strontium Crosslinked, Inhomogeneous Gelled Alginate Microspheres

### Radium-223 and Decay Daughter Radionuclides

In this part of Example 3, binding of Ra-223 and daughter nuclides to strontium crosslinked, inhomogeneous gelled alginate microspheres, and the stability of said binding, was examined.

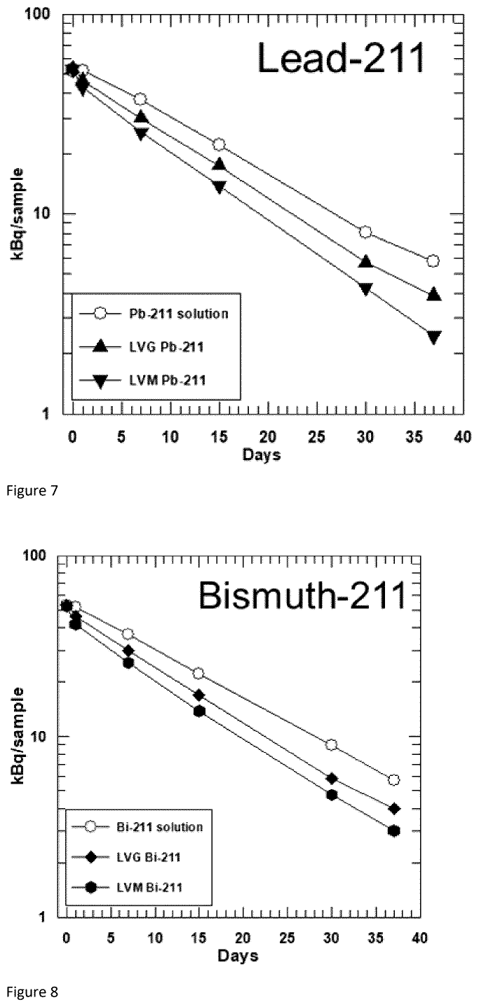

Alginate (LVG and LVM) at a 2% concentration in distilled water were gelled with Sr²⁺, using a gelling bath containing 50 mM SrCl₂ in distilled water, and no NaCl. The absence of non-gelling ions leads to an inhomogeneous gelling process. The microspheres were washed three times using PBS and then resuspended in 1 mL of PBS. A volume of radium-223 in equilibrium with its daughter radionuclides was added and incubated together with the microspheres for 60 minutes at room temperature. FIGS. 6-8 show the binding of radium-223 and decay daughter radionuclides lead-211 and bismuth-211 to the resulting microspheres. In the Figures, “Ra-223 solution” indicates the sample not containing alginate microspheres and is a control of the decay rate of the radionuclide alone. Radioactivity associated with the alginate microspheres and in the radionuclide solution was determined by high purity germanium detector equipment (HPGe). The resulting data, kBq/sample, was plotted against the day of measurement using GraFit software. The slope of the curve represents the radioactive decay of the radionuclide.

The curves of FIGS. 6-8 indicate that despite the change in gelling ion from calcium to strontium compared to Example 2, Ra-223 was still able to bind to the alginate gel microspheres. The results also indicate that the particles resulting from the inhomogeneous gelation process binds and retains radionuclide cations more efficiently than the homogeneous gelation process used in Example 2, as evidenced by the slopes of the radioactive decay curves are almost identical to the radionuclide alone decay curve. This is an indication that the microspheres made by the inhomogeneous gelation method do not release radium-223 or decay daughters over time. It is apparent also that decay daughter radionuclides are not released from the alginate network but are retained throughout radioactive decay.

### Actinium-225 and its Decay Daughter Radionuclides

In the second part of Example 3, the ability of strontium crosslinked alginate microspheres to bind 225-Ac and its decay daughters 221-Fr and 213-Bi were examined.

Homogeneous calcium crosslinked alginate microparticles were produced using PRONOVA LVG alginate (1%) as described in Example 1 wherein the gelling bath was composed of 50 mM SrCl₂ in purified water. The strontium crosslinked alginate microparticles were approximately 230 μm in diameter. Prior to use the alginate microparticles were washed in Dulbecco's phosphate buffered saline (D-PBS) to remove excess, non-crosslinked Sr²⁺ ions. For absorption of 225-Ac triplicate samples containing a known amount of strontium crosslinked alginate microparticles (approximately 270 mg in triplicate) were suspended in D-PBS (500 μL) containing approximately 2 kBq 225-Ac and incubated at room temperature for 1 hour. Following incubation, the microparticles were centrifuged, and the supernatant was removed to analyze for residual radioactivity. The microparticles were subsequently washed three times using 500 μL portions of D-PBS and finally resuspended in 500 L D-PBS, transferred to RIA tubes, and counted using a Cobra II NaI gamma counter. The gamma counter was setup with detector channels equivalent to total radioactive energy (15-2000 keV), 180-260 keV (221-Fr), and 400-500 keV (213-Bi).

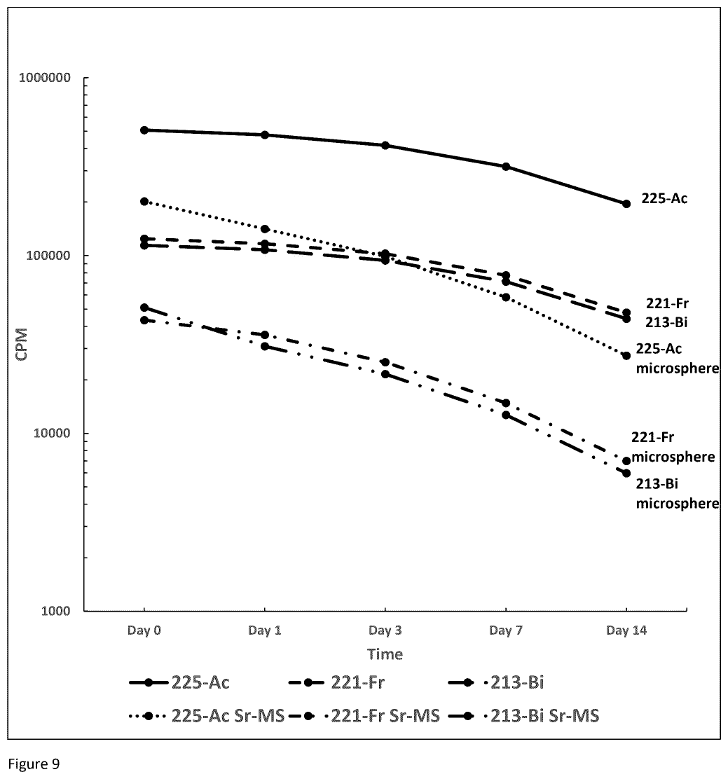

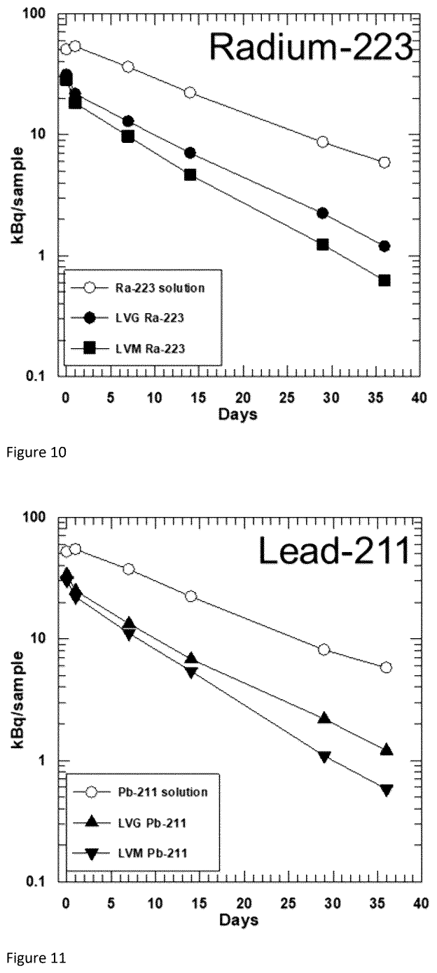

The results graphically displayed in FIG. 9 show: 1) that the 225-Ac solution used also contained the decay daughter radionuclides 221-Fr and 213-Bi; 2) that strontium crosslinked alginate microparticles were able to bind not only 225-Ac but also the decay daughter radionuclides 221-Fr and 213-Bi. The relative amounts of radionuclides bound was approximately 40% of the total radioactivity added. However, the bound radioactivity of strontium crosslinked alginate microparticles was not corrected for the amount of alginate present, i.e., of the approximately 270 mg alginate microparticles in each sample, the amount of alginate was only 1% resulting in a weight of alginate of 2.7 mg). The samples were stored at room temperature for 14 days and radioactivity was analyzed in D-PBS-washed samples. FIG. 9 also shows that the decay daughter radionuclides 221-Fr and 213-Bi are retained by the alginate microparticles. This indicates that the formation of 221-Fr from the decay of 225-Ac as well as the formation of 213-Bi from the subsequent decay of 221-Fr to 217-At (not detected due to weak gamma energy) and further to 213-Bi are both retained by the alginate microparticles and not released from the alginate network but are retained throughout radioactive decay. This demonstrates the chelating ability of alginate for 225-Ac and decay daughter radionuclides and retention of said decay daughters within the alginate microparticle.

### Example 4: Binding of Ra-223 and Daughter Nuclides to Barium Crosslinked, Inhomogeneous Gelled Alginate Microspheres

In this Example, binding of Ra-223 to barium crosslinked, inhomogeneous gelled alginate microspheres, and the stability of said binding, was examined.

Alginate (LVG and LVM) at a 2% concentration in distilled water were gelled with Ba²⁺, using a gelling bath containing 50 mM BaCl₂ in distilled water, and no NaCl. The absence of non-gelling ions leads to an inhomogeneous gelling process. The microspheres were washed three times using phosphate-buffered saline (PBS) and then resuspended in 1 mL of PBS. A volume of radium-223 in equilibrium with its daughter radionuclides was added and incubated together with the microspheres for 60 minutes at room temperature. FIGS. 10-12 show the binding of radium-223 and decay daughter radionuclides lead-211 and bismuth-211 to the resulting microspheres. In the Figures, the slope of the curve represents the radioactive decay of the radionuclide. “Ra-223 solution” indicates the sample not containing alginate particles, and is a control of the decay rate of the radionuclide alone. Radioactivity associated with the alginate microspheres and in the radionuclide solution was determined by high purity germanium detector equipment (HPGe). The resulting data, kBq/sample, was plotted against the day of measurement using GraFit software.

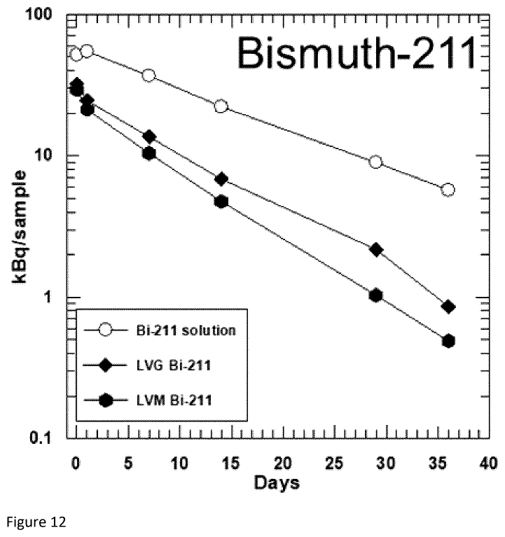

The results from Example 4 compared to those from Example 2 also indicate that a gel obtained by an inhomogeneous gelation process binds and retains radionuclide cations more efficiently than a gel obtained by a homogeneous gelation process as used in Example 2, as evidenced by the slope of the radioactive decay curves being almost identical to the radionuclide alone decay curve (FIG. 9). This is a further indication that the microspheres made by the inhomogeneous gelation method do not release radium-223 or decay daughters over time. Further, it is apparent from FIGS. 11-12 that decay daughter radionuclides lead-211 and bismuth-211 are also not released from the alginate network but are retained throughout radioactive decay.

In the Examples above, radium-223 and 225-Ac were used, however, similar conditions and results may be obtained using other radionuclides.

### Example 5: Binding of Lutetium-177 to Calcium Crosslinked, Inhomogeneous Gelled Alginate Microspheres

In this Experiment, binding of a trivalent radionuclide cation to alginate microspheres was studied.

Calcium cross-linked inhomogeneous alginate gel microspheres were produced from a 2% sodium alginate solution in de-ionised water (PRONOVA LVG sodium alginate) electrostatically delivered into a gelling bath composed of 50 mM CaCl₂) in de-ionised water. The pre-formed alginate gel microspheres (2.2 grams) were then washed with 0.9% NaCl then divided into three separate samples. The samples were then incubated with a solution of ¹⁷⁷Lu³⁺ (a beta emitting radionuclide) in 0.9% NaCl for 1 hour. After incubation, the alginate gel microspheres were washed three times with 0.9% NaCl before incorporated radioactivity was measured using a NaI detector. Results are given as un-corrected counts per minute.

TABLE 2
Binding efficiency of lutetium-177 to preformed
alginate microspheres (calcium cross-linked)

| | Available radioactivity | Radioactivity of |
|---|---|---|
| Sample | (CPM) | microspheres (CPM) |
| 1 | 2952690 | 1744735 |
| 2 | 2886965 | 1510923 |
| 3 | 2962539 | 1649520 |
| Average ± SD | 2934064 ± 41086 | 1635059 ± 117575 |
| Radioactivity bound (%) | | 55.7% |
| CPM/gram microspheres | | 1306351 |

As can be seen from Table 2, of the available amount of radioactivity, approximately 56% was bound to the pre-formed alginate gel microspheres as shown in Table 1 below. Hence, the gel was able to chelate a trivalent radionuclide cation.

### Example 6: Binding of Thorium-227 to Calcium Crosslinked, Inhomogeneous Gelled Alginate Microspheres

In this Experiment, binding of a tetravalent radionuclide cation to alginate microspheres was studied.

Calcium crosslinked, inhomogeneous gelled alginate microspheres were made in the same manner as shown in Example 5 above. The microspheres were washed three times using 0.9% NaCl solution, resuspended in 0.8% NaCl and incubated with a solution of ²²⁷Th⁴⁺. Of the available amount of radioactivity, approximately 30% bound to the pre-formed alginate microspheres (Table 3). Table 3 also shows that 100% of the available ²²³Ra, ²¹¹Pb and ²¹¹Bi produced during radioactive decay of ²²⁷Th (daughter radionuclides) bind to pre-formed alginate microspheres following 1 hour of incubation.

TABLE 3
Binding efficiency of thorium-227 and its decay daughters
radium-223, lead-211, and bismuth 211 to preformed
alginate microspheres (calcium cross-linked)

| | Available | Bound at 1 h | | Bound at 2 h | |
|---|---|---|---|---|---|
| Sample | radioactivity (Bq) | Bq | % | Bq | % |
| Thorium-227 | 81214 | 24679 | 30.4 | 30647 | 37.7 |
| Radium-223 | 9430 | 10512 | 111.5 | 11201 | 118.8 |
| Lead-211 | 9450 | 10850 | 114.8 | 10757 | 118.3 |
| Bismuth-211 | 11158 | 10067 | 90.2 | 11154 | 99.9 |

Additional experiments performed in the same manner as described above evaluated the binding of thorium-227 and decay daughters to guluronate-rich alginate (LVG) and to mannuronate-rich alginate (LVM) following a 2-hour incubation period in order to repeat the findings in Table 3. Inhomogeneous gelation was used to produce calcium cross-linked alginate microspheres. Equivalent amounts (mg) of microspheres were washed with 0.9% NaCl and then incubated with a solution of thorium-227 for 2 hours. The results are summarised below (Table 4) and show that 38.8% of the available 227Th bound to the G-rich (LVG) alginate gel microspheres, while 20.4% of available 227Th bound to alginate gel microspheres made from M-rich (LVM) alginate after a 2-hour incubation period.

TABLE 4
Binding of thorium-227 to G-rich and M-rich alginate microspheres

| | Radioactivity | % of available |
|---|---|---|
| Sample | (Bq ²²⁷Th) | ²²⁷Th |
| ²²⁷Th solution | 67300 | 100% |
| G-rich (LVG) microspheres | 26100 | 38.8% |
| M-rich (LVM) microspheres | 13800 | 20.4% |

It can be concluded that the alginate gel is able to bind a tetravalent radionuclide cation. The results of the latter experiment showed that 38.8% of available thorium-227 bound to the alginate microspheres after a 2-hour incubation period. The results shown in Table 3 showed that 37.7% of available thorium-227 bound to microspheres. The percent thorium-227 bound to alginate microspheres is essentially the same between the two experiments. The results are also consistent with the finding that G-rich alginate is able to chelate thorium-227 more efficiently than the M-rich alginate due to the increased number of guluronate monomers in the alginate polymer molecule. This is shown by the fact that while microspheres made using G-rich LVG alginate bound 37.7% of available thorium-227, only 20.4% of available thorium-227 was bound to microspheres made using M-rich LVM alginate

### Example 7: Calcium Alginate Microparticles Incorporate Radionuclide Cation and Retain Decay Daughter Nuclides

This example demonstrates that dry microparticles of calcium alginate can also bind radionuclides and retain daughter nuclides. Calcium alginate was produced via the conversion of sodium alginate powder suspended in isopropyl alcohol by adding an aqueous solution of calcium chloride as exemplified in US 2011/0053886 incorporated herein.

### Radium-224 and Decay Daughters

A 30 mg sample of calcium alginate microparticles, having an average size distribution (DV₅₀) of 13 μm, was resuspended first in 200 μl water, then 150 μl radium-224 solution was added followed by an additional 150 μl of water. The sample was vortex mixed then incubated at room temperature for 30 min. An aliquot of 500 μl water was added to the particle suspension. The sample was vortex mixed then centrifuged for 1 min at 6000 rpm. The supernatant was transferred to another Eppendorf tube labelled Wash 1. The particle sample was then resuspended in 1000 μl water, vortexed, then centrifuged. The supernatant was transferred to an Eppendorf tube labelled Wash 2. The wash with 1000 μl water was repeated and the supernatant collected to tube Wash 3. Samples were then analysed for radioactivity using a high purity germanium detector.

The sample was kept at room temperature and analysed again at day 1 (24 hours after labelling) and 12 days after labelling. Radioactivity was determined after first washing the particles with water. The results are shown in Table 5.

TABLE 5
Binding of radium-224 and decay daughters lead-212
and bismuth 212 to calcium alginate microparticles.

| | 224- | 212- | 212- | 208- | | | | | |
|---|---|---|---|---|---|---|---|---|---|
| | Ra | Pb | Bi | Tl | | 224- | 212- | 212- | 208- |
| Time | (Bq) | (Bq) | (Bq) | (Bq) | % | Ra | Pb | Bi | Tl |
| Radionuclide | 9210 | 9720 | 9140 | 9090 | % | 100 | 100 | 100 | 100 |
| Solution = | | | | | Available | | | | |
| Available | | | | | radioactivity | | | | |
| radioactivity | | | | | | | | | |
| Day 0 | 8740 | 8370 | 7640 | 7900 | Day 0% | 94.9 | 86.1 | 83.6 | 86.9 |
| particles | | | | | bound | | | | |
| directly after | | | | | radioactivity | | | | |
| labelling | | | | | relative to | | | | |
| | | | | | available | | | | |
| | | | | | radioactivity | | | | |
| Day 1 | 7270 | 7840 | 7650 | 7490 | Day 1 | 100 | | | |
| radioactivity | | | | | % radioactivity | | | | |
| associated | | | | | remaining | | | | |
| with | | | | | bound | | | | |
| particles | | | | | relative to | | | | |
| | | | | | theoretical | | | | |
| | | | | | radioactivity | | | | |
| | | | | | following | | | | |
| | | | | | decay | | | | |
| Calculated | 7232 | | | | | | | | |
| radioactivity | | | | | | | | | |
| based on t_{1/2} | | | | | | | | | |
| Day 12 | 913 | 1040 | 910 | 991 | Day 12% | 101 | | | |
| radioactivity | | | | | radioactivity | | | | |
| associated | | | | | remaining | | | | |
| with | | | | | bound | | | | |
| particles | | | | | relative to | | | | |
| | | | | | theoretical | | | | |
| | | | | | radioactivity | | | | |
| | | | | | following | | | | |
| | | | | | decay | | | | |
| Calculated | 900 | | | | | | | | |
| radioactivity | | | | | | | | | |
| based on t_{1/2} | | | | | | | | | |

The radioactive half-life of 224-Ra t_{1/2}=3.66 days; 212-Pb t_{1/2}=10.6 hours; 212-Bi t_{1/2}=60.5 minutes; 208-TI t_{1/2}=32 minutes. The solution of 224-Ra used was in secular equilibrium meaning that the decay daughter radionuclides 212-Pb, 212-Bi, and 208-TI are present in a constant amount in the solution. This solution represents the available radioactivity, and radionuclides, added to calcium alginate microparticles. Following incubation and a wash to remove unbound radioactivity, the radioactivity associated with the alginate microparticles was again measured respective to each decay daughter radionuclide. The % bound radioactivity relative to the available radioactivity was calculated. It can be seen that about 90% of the available radioactivity emanating from each radionuclide was bound to the alginate microspheres.

When the particles were washed on day 1 after labelling, and radioactivity determined (HPGe detector), in addition, the amount of 224-Ra radioactivity theoretically remaining associated with the alginate microparticles was calculated using an available radioactive decay calculator (http://www.radprocalculator.com). The calculation shows that of the original 8720 Bq 224-Ra associated with the alginate microparticles on Day 0 immediately after labelling, 7232 Bq 224-Ra would be present if the microparticles retained all bound radioactivity. The reduction in radioactivity from 8720 Bq to 7232 Bq represents the natural decay of 224-radium. Radioactivity associated with the alginate microparticles on Day 1 after labelling was measured to be 7270 Bq, so 100% of radioactivity was still associated with the microparticles adjusted for decay after 1 day.

Note: the daughter radionuclides 212-Pb, 2121-Bi and 208-TI are continuously being generated as a result of the radioactive decay of 224-Ra. Therefore, these data are not used to calculate % bound radioactivity. Finally, the natural decay of 224-Ra at day 12 after labelling was calculated to be 900 Bq, reduced from 8740 Bq. The measured radioactivity associated with the alginate microparticles was 913 Bq, which also is 100% of radioactivity adjusted for decay of 224-Ra. The conclusion is that the calcium alginate microparticles effectively bind radionuclides, including decay daughters and continues to bind the radionuclides.

### Actinium-225 and Decay Daughters

In the first experiment, 50 mg of calcium alginate microparticle samples in triplicate were resuspended in a solution containing 20 kBq of 225-Ac and incubated for 1 hour at room temperature. The samples were then centrifuged, and the supernatant transferred to individual RIA tubes. 500 μL of purified water was added to each sample and the particles were resuspended. The samples were centrifuged and the supernatant representing the wash was transferred to individual RIA tubes. Finally, 500 μL purified water was added to each calcium alginate microparticle sample and the resulting suspension was transferred to individual RIA tubes. All tubes were counted for gamma radioactivity using a Cobra II NaI gamma counter with detector channels equivalent to total radioactive energy (15-2000 keV), 180-260 keV (221-Fr), and 400-500 keV (213-Bi). Following the initial gamma count, the calcium alginate samples were further incubated at room temperature. The samples were centrifuged, supernatant collected, washed (also collected) and resuspended in purified water at specified time points up to 31 days.

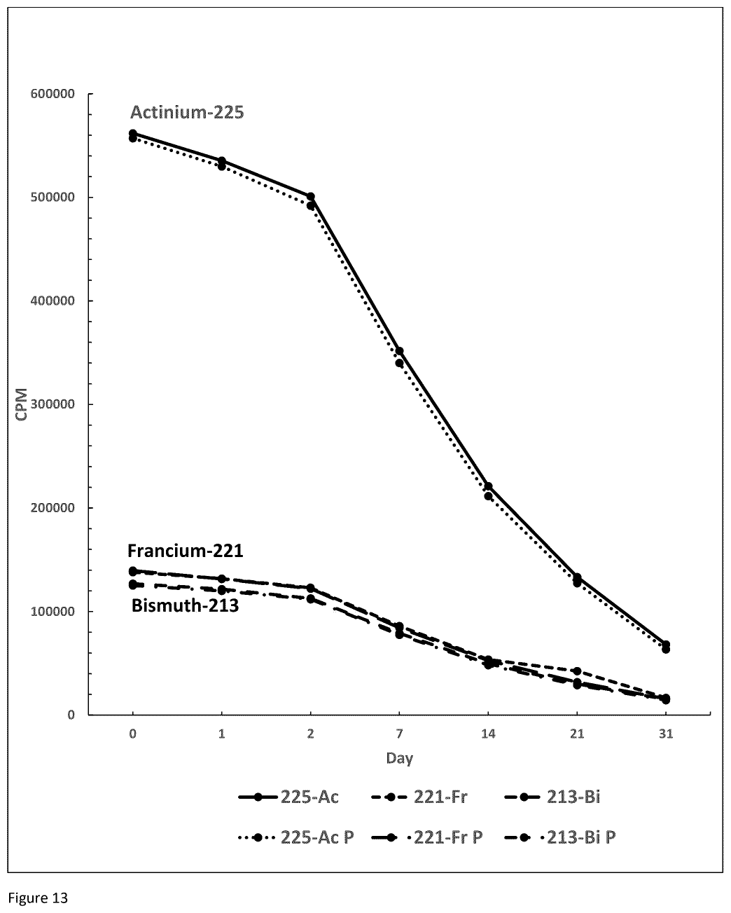

The results shown in FIG. 13 compare the radioactive counts (as CPM) associated with the calcium alginate microparticles to the radioactive counts of an equivalent 20 kBq solution of 225-Ac without microparticles. The first conclusion is that 225-Ac solution (without calcium alginate microparticles) is in equilibrium with the decay daughter radionuclides 221-Fr and 213-Bi. However, the radioactivity associated with calcium alginate microparticles also shows the presence of 225-Ac and the two decay daughter radionuclides 221-Fr and 213 Bi. This same pattern is seen for all time points, i.e., the calcium alginate microparticles bind 225-Ac and retain 221-Fr and 213 Bi formed from radioactive decay of 225-Ac. Since 213-Bi is generated from 217-At the data indicate that this decay daughter must also be retained by the calcium alginate microparticles even though it is not detected due to a very weak gamma energy. Another conclusion drawn from the data presented in FIG. 13 is that greater than 90% of available radioactivity is associated with the microparticles at all time points demonstrating that alginate is an effective chelator of 225-Ac and its decay daughter radionuclides.

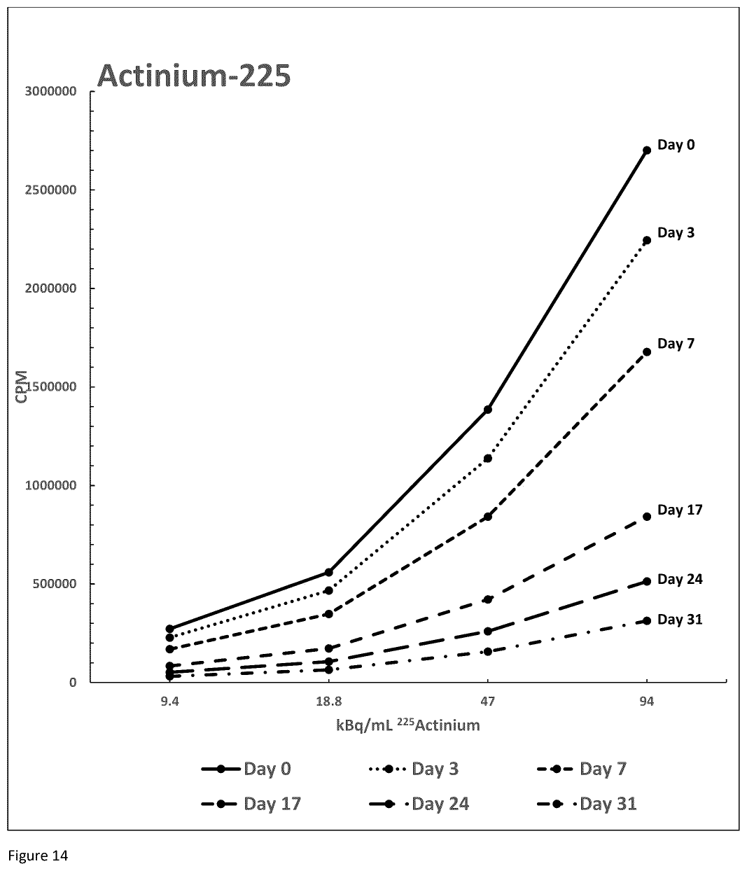

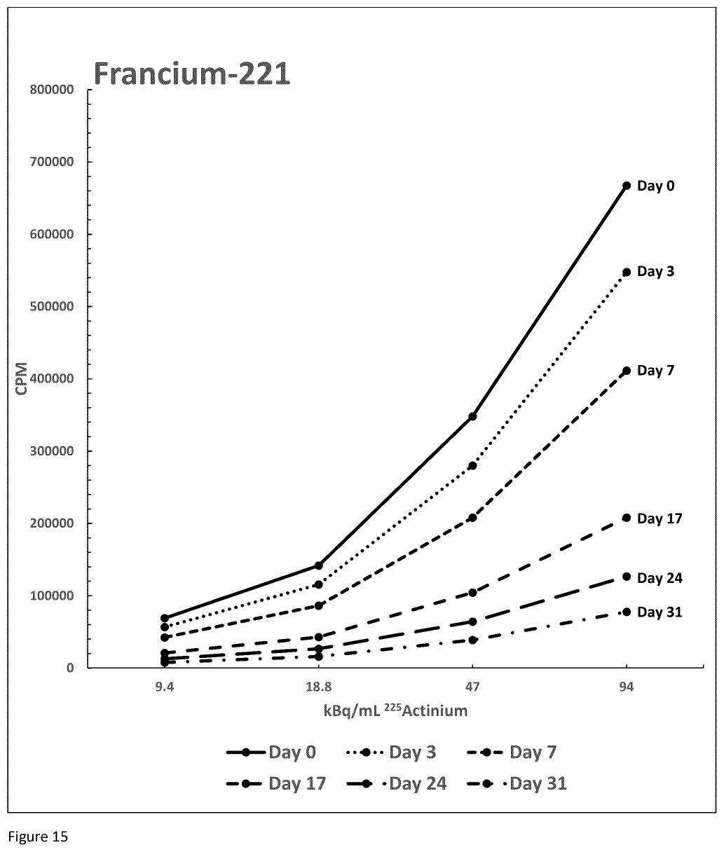

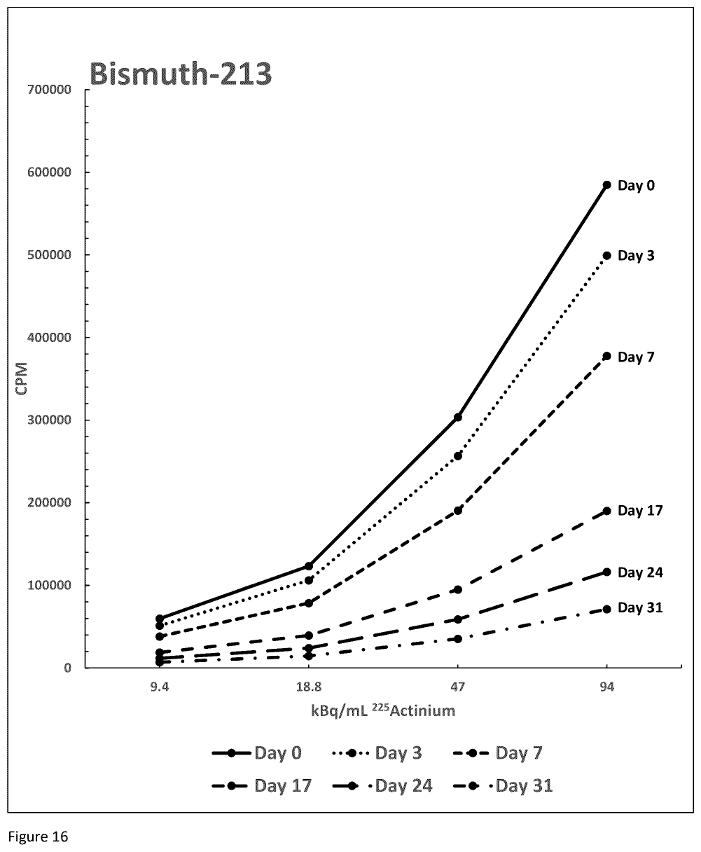

In a second experimental setup, 10 mg samples of calcium alginate microparticles were suspended in varying amounts (μL) of aqueous 225-Ac equivalent to approximately 9.4, 18.8, 47, and 94 kBq/mL in a total volume of 1000 UL and incubated at room temperature for 1 hour. The samples were then centrifuged, the supernatant transferred to RIA tubes and the calcium alginate pellet was resuspended in 1000 μL of purified water. After centrifugation, the resulting wash was also transferred to separate RIA tubes, and the pellet was resuspended in 1000 μL purified water and transferred to separate RIA tubes. All samples were counted using a Cobra II NaI gamma counter with detector channels equivalent to total radioactive energy (15-2000 keV), 180-260 keV (221-Fr), and 400-500 keV (213-Bi). Following the initial gamma count, the calcium alginate samples were further incubated at room temperature. The samples were centrifuged, supernatant collected, washed (also collected) and resuspended in purified water at specified time points up to 31 days. The results showing the counts (CPM) versus the amount of 225-Ac added are shown in FIG. 14 for 225-Ac (total radioactive counts), FIG. 15 for 221-Fr, and FIG. 16 for 213-Bi.

In all calcium alginate samples and for all time points, greater than 90% of available radioactivity was associated with the microparticles and less than 1% was associated with the supernatant or the wash. In addition, as the amount (volume) of 225-Ac added to the calcium alginate microparticles increased, i.e., the radioactive concentration increased, so did the amount of radioactivity associated with the microparticles. Since no plateau was reached for the uptake of radioactivity, the saturation of binding of 225-Ac and decay daughter radionuclides was not reached. There is, therefore, further capacity for calcium alginate microparticles to bind 225-Ac and its decay daughter radionuclides. This has a practical implication for formulating an amount of calcium alginate microparticles having sufficient therapeutic radioactivity.

### Example 8: Alginate Gel Stability

The purpose of this Example was to show that the degradability of an alginate gel can be changed based on the composition of the alginate gel.

Various formulations of alginate gels were cast in the wells of a 12-well tissue culture plate and allowed to stand for 2 hours at room temperature. The formulations consisted of different alginates: Formulation 3 used PRONOVA LVM sodium alginate; Formulation 24 used sterile PRONOVA SLM20 alginate; Formulation 25 used PRONOVA LVG alginate. The alginates were dissolved in de-ionised water to give a 2% solution. The gelling ion was calcium dissolved in de-ionised water. The gels were weighed at day 0 and each placed in a separate pre-weighed 50 mL plastic centrifuge tube marked with the formulation number, three gels for each formulation, one gel in each centrifuge tube. Twenty mL of 0.9% NaCl solution was added to each centrifuge tube and the tubes were placed in a shaking incubator at 30° C. at 100 rpm. The 0.9% NaCl solution was changed each day. The gels were weighed at various days after preparation by removing the NaCl solution and weighing the tubes including the gels. The weight of the gel was calculated by subtracting the weight of the tube from the tube+gel weight. The data points in FIG. 12 represent the average of 3 gels for each formulation.

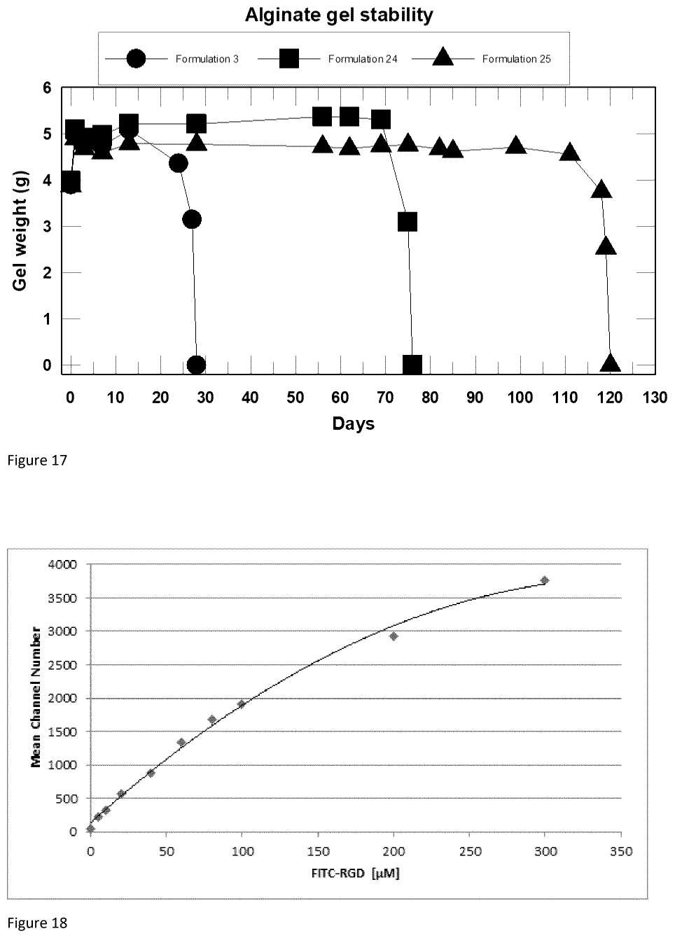

### With Reference to FIG. 17:

Formulation 3 (PRONOVA LVM): It took 24 days before the gels in formulation 3 dissolved. By day 28 the gels were completely dissolved.

Formulation 24 (PRONOVA SLM20): It took 75 days before the gels in formulation 24 dissolved and by day 76 the gels were completely dissolved.

Formulation 25 (PRONOVA LVG): It took 118 days before the gels in formulation 25 dissolved and by day 120 the gels were completely dissolved.

The results show that the stability of the alginate gel can be modified by changes in the formulation. G-rich alginate formed a gel with the longest stability, which the M-rich alginate LVM resulted in the shortest stability. PRONOVA SLM20 is a sterile freeze-dried alginate. This alginate also formed gels and resulted in a stability between those of the LVM and LVG alginates.

### Example 9—Binding of FITC-RGD to MDCK Cells

This example was performed in order to investigate the binding of the RGD peptide to cells.

MDCK cells (Madin Darby canine kidney, ATCC CCL-34) were trypsinised and counted before a volume corresponding to 500,000 cells were transferred to 15 mL centrifuge tubes. The cells were centrifuged, and the supernatant removed before FITC-RGD peptide was added to the cells. The cells were incubated for 30 minutes at 37° C. before they were centrifuged, and the supernatant was removed. The cells were washed three times with PBS and centrifuged between each wash before 1 mL sheath fluid was added to the cells and the samples were analysed with flow cytometry. FIG. 18 shows the Mean Channel Number versus the concentration of FITC-RGD peptide added to the cells. The Mean Channel Number (y-axis) is a representation of the fluorescence measured from FITC-RGD peptide and is directly related to the amount of FITC-RGD peptide bound to cells. The Figure shows that as the amount of FITC-RGD peptide increases the fluorescence measured appears to level off, i.e., reach a plateau. This indicates that concentrations of FITC-RGD peptide over 300 M saturate all available cell surface binding sites for RGD. Other experiments (not shown) demonstrate that the binding of

FITC-RGD peptide was specific to cell receptors able to bind the RGD peptide.

### Example 10: Demonstration of Biological Functionality of RGD-Alginate

The aim of the Experiment was to investigate the biological functionality of RGD-alginate, in particular its binding to cells.

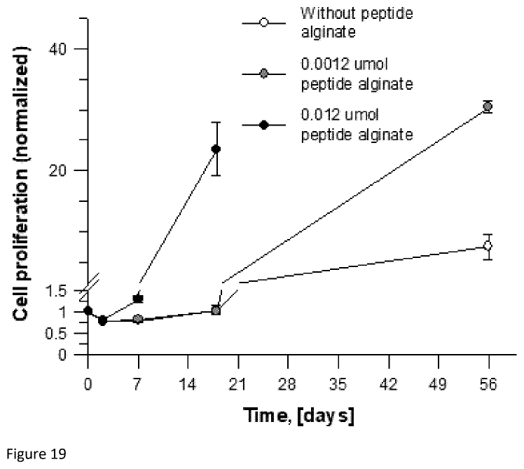

A dose-response type of experiment was set up where C₂C₁₂ mouse myoblasts (ATCC CRL-1772) were seeded into a 3D cell culture system. The cells were immobilised in the 3D cell culture system with alginate and peptide-coupled alginate (PRONOVA UP LVG and NOVATACH RGD) to a final alginate concentration of 0.5%. Cells were exposed to sodium alginate without peptide alginates, with 0.012 μmol peptide-coupled alginate, or with 0.0012 μmol peptide-coupled alginate. Changes in cell number over time were determined by trypsinising cells and counting using a phase contrast microscope and hemocytometer. These data are shown in FIG. 19. Each data point is the average of from three individual discs (N=3). Error bars represent standard error.

C₂C₁₂ mouse myoblast cells are considered anchorage dependent, i.e., the cells need to be attached to a substrate in order to proliferate. Integrins are cellular receptors responsible for cell adhesion and the RGD sequence is the most common peptide motif responsible for cell adhesion to the extracellular matrix (ECM). The RGD peptide sequence is found within many matrix proteins, including fibronectin, fibrinogen, vitronectin, osteopontin, and several other adhesive extracellular matrix proteins. Without the interaction between integrin receptors and a ECM substrate cells will not proliferate. This is especially apparent when cells are cultured in a 3-dimensional cell culture system that does not provide ECM or adhesion proteins. Therefore, an increase in cell proliferation was used as a measure of RGD-alginate binding to cells that displayed little to no increase in proliferation when cultured in vitro. All samples containing RGD-alginate showed a measurable increase in cell number by Day 14, and the results appeared in a dose-dependent manner. Hence, it can be concluded that the RGD-alginate interacts with receptors on the cells for cell adhesion resulting in signals for cell adhesion which leads to the stimulation of cell proliferation when cells were grown in 3-dimensional cultures.

1. An alginate gel comprising
an alginate comprising at least one peptide having a peptide sequence for interacting with a receptor of a cell affected by a proliferative disease;
at least one type of divalent cations selected from the group comprising Ba²⁺, Sr²⁺, Ca²⁺, Cu²⁺, Zn²⁺, and combinations thereof; and
at least one radionuclide cation selected from the group comprising actinium-225, actinium-228, astatine-211, barium-140, bismuth-210, bismuth-211, bismuth-212, bismuth-213, calcium-45, calcium-47, copper-64, francium-221, gallium-67, holmium-166, indium-111, iridium-192, iron-59, lead-211, lead-212, lead-214, lutetium-177, osmium-191, osmium-193, palladium-103, platinum-197, radium-223, radium-224, radium-225, rhenium-186, rhenium-188, samarium-153, scandium-46, scandium-47, silver-111, strontium-85, strontium-89, tellurium-129, tellurium-132, terbium-160, terbium-161, thallium-201, thallium-206, thallium-210, thorium-227, thorium-231, thorium-234, tin-113, tungsten-185, tungsten-187, vanadium-48, ytterbium-169, yttrium-88, yttrium-90, yttrium-91, zirconium-95, and combinations thereof,
wherein the alginate chelates the divalent cations and the at least one radionuclide cation.

2. The alginate gel according to claim 1, wherein the peptide sequence is selected from the group comprising integrin-binding peptide sequences, LDL-binding peptide sequences, MMP-2-binding peptide sequences, IL13R2a-binding peptide sequences, VDAC1-binding peptide sequences, NBD-binding peptide sequences, cMYC-binding peptide sequences, CXCR4-binding peptide sequences, MDGI-binding peptide sequences, and combinations thereof.

3. The alginate gel according to claim 1, wherein the peptide is selected from the group of RGD, TFFYGGSRGKRNNFKTEEY, MCMPCFTTDHQMARKCDDCCGGKGRGKCYGPQCLCR, ACGEMGWGWVRCGGSLCW, SWTWEKKLETAVNLAWTAGNSNKWTWK, TALDWSWLQTE, WPGSGNELKRAFAALRDQI, RACRFFC, ACGLSGLGVA, and RPKPQQFFGLM.

4. The alginate gel according to claim 2, wherein the peptide sequence is arginine-glycine-aspartic acid (RGD).

5. The alginate gel according to claim 1, wherein the alginate gel is in the form of a particle.

6. The alginate gel according to claim 1, wherein the at least one radionuclide cation is located less than 100 μm below the surface of the alginate gel, preferably less than 50 μm below the surface of the alginate gel, more preferably less than 10 μm below the surface of the alginate gel.

7. A method for providing at least one alginate gel particle comprising an alginate comprising at least one peptide having a peptide sequence for interacting with a receptor of a cell affected by a proliferative disease and at least one radionuclide cation, wherein the method comprises the steps of
i) providing a solution of an alginate, wherein the alginate comprises at least one peptide having a peptide sequence for interacting with a receptor of a cell affected by a proliferative disease;
ii) dripping the solution from step i) into an aqueous solution comprising a divalent cation, wherein said divalent cation is selected from Ca²⁺, Sr²⁺, and Ba²⁺, to form at least one alginate gel particle; and
iii) contacting the alginate gel particle of step ii) with a solution comprising at least one radionuclide cation.

8. A composition comprising an alginate gel according to claim 1 together with at least one pharmaceutically acceptable carrier, diluent, and/or excipient.

9. A kit comprising,
in a first container,
an alginate gel comprising
an alginate comprising
at least one peptide having a peptide sequence for interacting with a receptor of a cell affected by a proliferative disease; and
at least one type of divalent cations selected from the group comprising Ba²⁺, Sr²⁺, Ca²⁺, Cu²⁺, Zn²⁺, and combinations thereof; and
optionally, a suitable solvent; and
in a second container,
at least one radionuclide cation, and
a suitable solvent.

10. A kit comprising,
in a first container,
a water-soluble alginate,
wherein the alginate comprises at least one peptide having a peptide sequence for interacting with a receptor of a cell affected by a proliferative disease,
water, and
optionally, another suitable solvent;
in a second container,
particles that are insoluble in water, said particles comprising an alginate and at least one type of divalent cation selected from the group comprising Ba²⁺, Sr²⁺, Ca²⁺, Cu²⁺, Zn²⁺, and combinations thereof, and
water; and
in a third container
at least one radionuclide cation, and
a suitable solvent.

11. The gel according to claim 1 for use as a medicament.

12. A method for the treatment of a proliferative disease of a subject, the method comprising a step of administering the gel according to claim 1 to the subject.

13. The method according to claim 12, wherein said proliferative disease is a cancer, a hyperplastic or neoplastic disease.

14. (canceled)

15. The method according to claim 13 wherein the gel is administered into a cavity of the subject where cancerous tissue has been removed.

16. The composition according to claim 8 for use as a medicament in the treatment of a proliferative disease.

17. The composition according to claim 16 wherein the proliferative disease is a cancer, a hyperplastic or neoplastic disease.

18. A method for the treatment of a proliferative disease of a subject, the method comprising a step of administering the composition according to claim 8 to the subject.

19. The method according to claim 17 wherein said proliferative disease is a cancer, a hyperplastic or neoplastic disease.

20. The method according to claim 18 wherein the composition is administered into a cavity of the subject where cancerous tissue has been removed.


SEQUENCE LISTING (27 sequences)

SEQ ID NO: 1 - protein, 5 aa, Homo sapiens
  SITE 4: note = D-phenylalanine
  MOD_RES 1..5: note = cyclic peptide
RGDFK

SEQ ID NO: 2 - protein, 19 aa, Homo sapiens
TFFYGGSRGKRNNFKTEEY

SEQ ID NO: 3 - protein, 36 aa, Homo sapiens
MCMPCFTTDHQMARKCDDCCGGKGRGKCYGPQCLCR

SEQ ID NO: 4 - protein, 18 aa, Homo sapiens
ACGEMGWGWVRCGGSLCW

SEQ ID NO: 5 - protein, 27 aa, Homo sapiens
SWTWEKKLETAVNLAWTAGNSNKWTWK

SEQ ID NO: 6 - protein, 11 aa, Homo sapiens
TALDWSWLQTE

SEQ ID NO: 7 - protein, 19 aa, Homo sapiens
WPGSGNELKRAFAALRDQI

SEQ ID NO: 8 - protein, 7 aa, Homo sapiens
RACRFFC

SEQ ID NO: 9 - protein, 10 aa, Homo sapiens
ACGLSGLGVA

SEQ ID NO: 10 - protein, 12 aa, Homo sapiens
YHWYGYTPQNVI

SEQ ID NO: 11 - protein, 12 aa, Homo sapiens
YHWYGYTPENVI

SEQ ID NO: 12 - protein, 12 aa, Homo sapiens
YHWYGYTPQDVI

SEQ ID NO: 13 - protein, 12 aa, Homo sapiens
YHWYGYTPKNVI

SEQ ID NO: 14 - protein, 12 aa, Homo sapiens
YHWYGYTPQKVI

SEQ ID NO: 15 - protein, 7 aa, Homo sapiens
KLARLLT

SEQ ID NO: 16 - protein, 7 aa, Homo sapiens
  MOD_RES 1..7: note = Cyclic peptide
KLARLLT

SEQ ID NO: 17 - protein, 11 aa, Homo sapiens
RPKPQQFFGLM

SEQ ID NO: 18 - protein, 6 aa, Homo sp.
GRGDSP

SEQ ID NO: 19 - protein, 9 aa, Homo sapiens
GGGGRGDSP

SEQ ID NO: 20 - protein, 8 aa, Homo sapiens
  SITE 1: note = D-phenylalanine
  SITE 4: note = D-tryptophan
  MOD_RES 2..7: note = cyclic peptide
  MOD_RES 8: note = ol at the end of the peptide
FCFWKTCT

SEQ ID NO: 21 - protein, 10 aa, Homo sapiens
  SITE 1: note = D-pyroglutamic acid
  SITE 6: note = D-lysine
EHWSYKLRPG

SEQ ID NO: 22 - protein, 9 aa, Homo sapiens
  SITE 1: note = D-tyrosine
  SITE 6: note = beta-alanine
  SITE 9: note = x=norleucine
YQWAVAHFX

SEQ ID NO: 23 - protein, 28 aa, Homo sapiens
HSDAVFTDNYTRLRKQMAVKKYLNSILN

SEQ ID NO: 24 - protein, 6 aa, Homo sp.
RRPYIL

SEQ ID NO: 25 - protein, 8 aa, Homo sp.
  SITE 1: note = D-Glutamic acid
EAYGWMDF

SEQ ID NO: 26 - protein, 8 aa, Homo sapiens
  SITE 1: note = x=norleucine
  MOD_RES 1: note = Acetyl at the N' terminus
  SITE 4: note = D-phenylalanine
XDHFRWGK

SEQ ID NO: 27 - protein, 36 aa, Homo sp.
YPSKPDFPGEDAPAEDLARYYSALRHYINLITRPRY